(19) United States
(12) Patent Application Publication
(10) Pub. No.: US 2023/0406873 A1
(43) Pub. Date: Dec. 21, 2023

(54) PROBES, SYSTEMS, AND METHODS FOR MAGNETIC RESONANCE PH SENSING

(71) Applicant: The General Hospital Corporation, Boston, MA (US)

(72) Inventors: Eric M. Gale, Charlestown, MA (US); Hannah S. Shafaat, Columbus, OH (US); Peter Caravan, Charlestown, MA (US); Huan Wang, Boston, MA (US)

(21) Appl. No.: 18/036,579

(22) PCT Filed: Nov. 16, 2021
(86) PCT No.: PCT/US2021/059500

§ 371 Date: May 11, 2023

Related U.S. Application Data
(60) Provisional application No. 63/114,955, filed on Nov. 17, 2020.

(51) Int. Cl. C07F 15/02; A61K 49/10

(52) U.S. Cl. CPC C07F 15/025; A61K 49/103

(57) ABSTRACT

The present disclosure relates to magnetic resonance imaging (MRI) using biochemically responsive imaging probes that can be used in, for example, non-invasive pH mapping in cancer and/or diseases characterized by aberrant metabolism using contrast enhanced MRI.

### CROSS-REFERENCE TO RELATED APPLICATIONS

This application claims the benefit of U.S. Provisional Application No. 63/114,955, filed Nov. 17, 2020, the entire disclosure of which is incorporated herein by reference.

### STATEMENT REGARDING FEDERALLY SPONSORED RESEARCH

This invention was made with government support under grant numbers DK120663 and GM128852 awarded by the National Institute of Health. The government has certain rights in the invention.

### TECHNICAL FIELD

The present disclosure relates to magnetic resonance imaging (MRI) using biochemically responsive imaging probes that can be used in, for example, non-invasive pH mapping in cancer and/or diseases characterized by aberrant metabolism using contrast enhanced MRI.

### BACKGROUND

Magnetic resonance imaging (MRI) using biochemically responsive imaging probes offers a potentially powerful approach to non-invasively detect, quantify, and map pathology at the molecular level. Biochemically responsive MR imaging probes can be detected via T₁-relaxation, chemical exchange saturation transfer (CEST), or direct nuclear observation.

The Gd complexes are commonly pursued as biochemically responsive T₁-relaxaton agents as they are detected with high sensitivity at concentrations that are safely achieved in vivo. However, the biochemical response from Gd-based relaxation agents is often limited by poor dynamic range. Gd complexes possess high relaxivity (r₁) even prior to probe activation and because MR signal in T₁-weighted images in vivo reflects both r₁ and probe concentration, it is challenging to develop Gd-based probes where r₁ is large enough so that interpretation of MR signal response is not confounded by uncertainty in tissue concentration. Probes that modulate MR signal through biochemically triggered changes in the chemical exchange saturation transfer (CEST) effect or ¹H or ¹⁹F chemical shift can provide an “off/on” effect, but low detection sensitivity is a major barrier to use.

Accordingly, there is a need to develop biochemically responsive MR relaxation agents as imaging probes where the biological stimuli promotes a large change in the probe's relaxivity. Disclosed herein are stable iron complex that switch between high-spin (S=5/2) monomeric and antiferromagnetically coupled dimeric species within the range of pH values encountered within human pathophysiology and at concentrations readily achieved with a typical dose of MR imaging probe. In these iron complexes, the dimeric antiferromagnetic species exhibits low relaxivity, whereas the corresponding monomeric S=5/2 complex exhibits high relaxivity.

### SUMMARY

Provided herein are compounds of Formula (I):

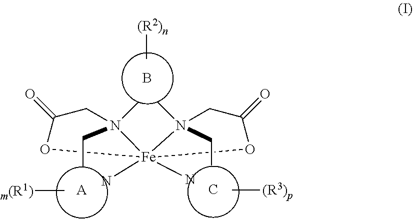

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)Rc, 4-6 membered heterocycloalkyl, 5-6 membered heteroaryl, C₃₋₆ cycloalkyl, —(C₁₋₆ alkyl)_{q}SO₂R^{A}, —(C₁₋₆ alkl)_{q}NHSO₂R^{A}, —(C═O)_{q}NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein is a compound of Formula (II):

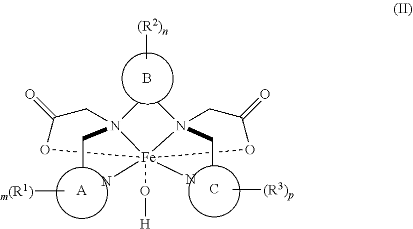

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein is a compound of Formula (III):

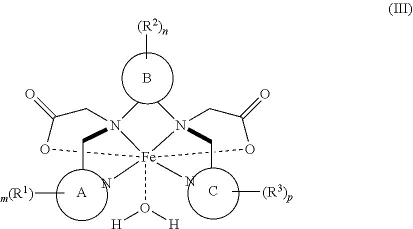

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alky)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein is a compound of Formula (IV):

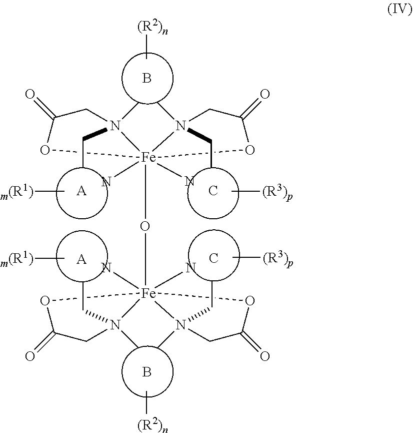

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}NR^{A}, (C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Some embodiments provide a method for in vivo imaging of a subject comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time. Step (b) comprises obtaining an image of an entire subject (e.g., a full body scan) as well as imaging specific regions of the subject's body. The subject is positioned in a magnet system configured to generate a polarizing magnetic field about at least a portion of a subject. A plurality of gradient coils configured to apply a gradient field to the polarizing magnetic field are energized. A radio frequency (RF) system configured to apply an excitation field to the subject is controlled to acquire magnetic resonance (MR) image data therefrom, and an image of the region of interest is reconstructed from the MR image data.

Some embodiments provide a method for detecting one or more regions of a subject having aberrant pH levels comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time. Regions of the subject having aberrant pH levels that contact the compound result in a modified longitudinal relaxation period of the compound that is reflected in the image.

Some embodiments provide a method of magnetic resonance (MR) imaging a tumor in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method of magnetic resonance (MR) imaging a blood clot in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method of magnetic resonance (MR) imaging a brain lesion in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method for detecting the presence or absence of a solid tumor in a subject comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method for determining the growth rate of a solid tumor in a subject having a solid tumor comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of a disrupted blood-brain-barrier in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of arterial stenosis in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of spinal stenosis in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for in vivo imaging of a subject comprising (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time. Step (b) comprises obtaining an image of an entire subject (e.g., a full body scan) as well as imaging specific regions of the subject's body. The subject is positioned in a magnet system configured to generate a polarizing magnetic field about at least a portion of a subject. A plurality of gradient coils configured to apply a gradient field to the polarizing magnetic field are energized. A radio frequency (RF) system configured to apply an excitation field to the subject is controlled to acquire magnetic resonance (MR) image data therefrom, and an image of the region of interest is reconstructed from the MR image data.

Some embodiments provide a method for detecting one or more regions of a subject having aberrant pH levels comprising ((a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time. Regions of the subject having aberrant pH levels that contact the compound result in a modified longitudinal relaxation period of the compound that is reflected in the image.

Some embodiments provide a method of magnetic resonance (MR) imaging a tumor in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method of magnetic resonance (MR) imaging a blood clot in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method of magnetic resonance (MR) imaging a brain lesion in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method for detecting the presence or absence of a solid tumor in a subject comprising (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method for determining the growth rate of a solid tumor in a subject having a solid tumor comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of a disrupted blood-brain-barrier in a subject (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of arterial stenosis in a subject comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of spinal stenosis in a subject comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

### DESCRIPTION OF DRAWINGS

FIG. 1. Shows the liquid chromatography (LC) trace of PyCy2AI at 254 nm detection (lower trace) and MS chromatogram of extracted m/z⁺402 (upper trace).

FIG. 2. Shows the LC trace of isolated Fe-PyCy2AI-Me at 254 nm detection (lower trace) and MS chromatogram of extracted m/z⁺455 (upper trace).

FIG. 3. Shows the LC trace of PyCy2AI-Me at 254 nm detection (lower trace) and MS chromatogram of extracted m/z⁺416 (upper trace).

FIG. 4. Shows LC trace of PyCy2AI-Me at 254 nm detection (lower trace) and MS chromatogram of extracted m/z⁺469 (upper trace).

FIG. 5. Shows HPLC trace of Fe-PyCy2AI at 254 nm detection recorded within 30 min (front trace) and 24 hours after (offset trace) of dissolution in buffered solutions of A: pH 4.2 (100 mM phosphate), B: pH 7.4 (100 mM phosphate), C: pH 9.0 (100 mM borate), and D: pH 11.2 (100 mM borate).

FIG. 6. Shows UV-vis spectra of Fe-PyCy2AI recorded at pH 4.2 and pH 10.6 at 310 K.

FIG. 7. Shows pH dependence on UV-vis absorbance of 0.1 mM of Fe-PyCy2AI between 280-380 nm at 310 K. The arrows denote 312 nm and 342 nm absorbance increase with increasing pH.

FIG. 8. Shows UV-vis of different concentrations of Fe-PyCy2AI between 280 and 380 nm at pH 9.0 (100 mM borate buffer) and 310 K. The arrows denote 312 nm and 342 nm absorbance increase with increasing concentration

FIG. 9. Shows the absorbance at 342 nm of different concentrations of Fe-PyCy2AI at pH 9.0 (100 mM borate buffer) and 310 K.

FIG. 10. Shows change in UV-vis absorbance at 348 nm after diluting a 5.0 mM aliquot of pH 7.4 solution of Fe-PyCy2AI to 0.5 mM in pH 5 buffer (100 mM phosphate) at 310 K.

FIG. 11. Shows change in UV-vis absorbance at 348 nm after diluting 5.0 mM aliquot of pH 5.0 solution of 5.0 mM Fe-PyCy2AI to 0.5 mM in pH 9.0 buffer (100 mM borate) at 310 K.

FIG. 12. Shows change in UV-vis absorbance at 348 nm after diluting a 5.0 mM aliquot of pH 9.0 Fe-PyCy2AI solution to 0.1 mM in pH 8.0 or pH 9.0 buffered solutions (100 mM borate) at 310 K.

FIG. 13. Shows change in UV-vis absorbance at 348 nm after adding a 5.0 mM aliquot of pH 5.0 solution of 0.1 mM Fe-PyCy2AI to a pH 8.5 or pH 9.0 buffered solution (100 mM borate) at 310 K.

FIG. 14. Shows pH titration curves of 7.0 mM H₂PyCy2AI•2TFA in the presence and absence of 1 molar equivalent Fe³⁺ at 310 K, I=0.1 M NaCl.

FIG. 15. Shows the EPR signal at different pHs of 1 mM Fe-PyCy2AI (9.38 GHz, 10 K, 0.2 mW). The arrows denote the signal intensity decrease with increasing pH.

FIG. 16. Shows the EPR signal intensity at 150 mT at different pHs of 1 mM Fe-PyCy2AI.

FIG. 17. Shows a CW EPR spectrum recorded with 1 mM concentration of Fe-PyCy2AI at T=10 K and Pμw=0.2 mW at pH 4.2.

FIG. 18. Shows a CW X-band EPR spectrum recorded with 1 mM concentration of Fe-PyCy2AI at T=10 K and Pμw=0.2 mW at pH 9.0.

FIG. 19. Shows experimental data (black line) overlaid with the total simulation (gray line) for pH=9.0.

FIG. 20. Shows high field power dependence of Fe-PyCy2AI at pH=7.0. CW X-band EPR spectra were recorded at T=20 K, and a concentration of 1 mM. The spectra are offset for clarity.

FIG. 21. Shows high field CW X-band EPR Spectra of 1 mM Fe-PyCy2AI at pH=7.0 at Pμw=0.002 mW and designated temperatures. The spectra are offset for clarity.

FIG. 22. Shows ORTEP diagram of (ML)₂O showing 50% thermal probability ellipsoids for all non-H atoms. Water solvent molecules are omitted for clarity.

FIG. 23. Shows bulk magnetic susceptibility at different pHs of 10 mM Fe-PyCy2AI at 310 K.

FIG. 24. Shows bulk magnetic susceptibility at different temperatures of 10 mM Fe-PyCy2AI at pH 5.0 and pH 9.0.

FIG. 25. Shows bulk magnetic susceptibility at different pHs of 10 mM Fe-PyCy2AI and 10 mM Fe-PyCy2AI-Me at 310 K.

FIG. 26. Shows overlay of Fe-PyCy2AI r₁ (black circles) and the Fe-PyCy2AI protonation state as a function of pH at 7.0 mM Fe concentration. Fe-I-IPyCy2AI speciation (dashed trace beginning at about 1.35 r₁), Fe-PyCy2AI speciation (black solid trace), (Fe-PyCy2AI)₂O (dotted trace), depronated species tentatively assigned as double deprotonated Fe-PyCy2AI (MLH-2; dashed trace ending at about 100% speciation).

FIG. 27. Shows ri of Fe-PyCy2AI at 310 K and 1.4 T as a function of Fe concentration.

FIG. 28. Shows ri of 0.2 mM and 2.0 mM solutions of Fe-PyCy2AI in human blood plasma at 1.4 T and 310 K as a function of pH.

FIG. 29. Shows plots of r₁ vs. pH of 7.0 mM Fe-PyCy2AI as pH is increased from pH 3.8 to pH 8.8, and then titrated in the reverse direction.

FIG. 30. Shows ri of 7.0 mM and 0.5 mM solutions of Fe-PyCy2AI as functions of pH.

FIG. 31. Shows the r₁ of Fe-PyCy2AI at pH 9.0, 310 K, and 4.7 T as a function of Fe concentration.

FIG. 32. Shows the r₁ of Fe-PyCy2AI at pH 9.0, 310 K, and 11.7 T as a function of Fe concentration.

FIG. 33. Shows r₁ of 10 mM Fe-PyCy2AI and 10 mM Fe-PyCy2AI-Me at 310 K and 1.4 T as a function of pH.

FIG. 34. Shows r₁ of Fe-PyCy2AI and Fe-PyCy2AI-Me at 310 K and 1.4 T as a function of Fe concentration.

FIG. 35. Shows T1-weighted 2D spin echo images (TR=250 ms, TE=6.33 ms, FA=90°) of phantoms containing 2.0 mM Fe-PyCy2AI adjusted to different pH values of 6.0-7.5, 4.7 T, 310 K.

FIG. 36. Shows T₂-relaxivity of water ¹⁷O (r₂^{O}) in the presence of 75 mM Fe-PyCy2AI at pH 5.0 or pH 6.6 as a function of temperature.

FIG. 37. Shows r₂^{O} at 310 K of Fe-PyCy2AI as a function of pH.

FIG. 38. Shows ¹⁷O T₂-relaxation rate as a function of temperature normalized to the mole fraction of water molecules coordinated to Fe³⁺ (R₂ᵣ) assuming q=1 at pH 5.0 and q=0.23 at pH 6.6.

FIG. 39. Shows T₂-relaxivity of water ¹⁷O (r₂^{O}) in the presence of Fe-PyCy2AI at pH 5.0, Fe-PyCy2AI at pH 6.6, and Fe-DTPA at pH 7.4.

### DETAILED DESCRIPTION

One embodiment provides a new class of dimeric Fe complex (Fe-PyCy2AI)₂O that changes T1-relaxivity (MR signal generating potency) in response to pH change. The two Fe(III) ions of the dimeric complex may be antiferromagnetically coupled resulting in low relaxivity. As pH decreases the complex breaks into two monomeric high-spin Fe(III) complexes which possess high relaxivity.

The complex may be rationally designed to detect pH in the pH 6.0-7.4 range that is relevant to human pathophysiology. A goal of the disclosure is to enable non-invasive pH mapping using contrast enhanced MRI. Acidosis in the pH 6.0-7.4 range occurs in solid tumors and other disease states characterized by aberrant metabolism. Reduced pH is associated with increased malignancy and resistant to chemo- and radiation therapy. The capability to non-invasively map tissue pH could aid in prognosis, treatment planning, and monitoring of treatment response in cancer and other diseases.

The disclosure provides rational design principals applied to (Fe-PyCy2AI)20 design, (Fe-PyCy2AI)₂O synthesis, as well as (Fe-PyCy2AI)2O characterization by pH-potentiometry, ¹H relaxometry, ¹⁷O relaxometry, UV-vis spectroscopy, bulk magnetic susceptibility measurements, electron paramagnetic resonance spectroscopy, and X-ray crystallography.

Described embodiments may modulate MR signal intensity in response to pH change. The complex is optimized to detect pH change in the pH 6.0-7.4 range that is relevant to human pathophysiology. The complex is detected with sensitivity close to that commercial gadolinium based MRI contrast agents. The complex can be detected with far greater sensitivity than pH imaging probes detected through chemical exchange saturation transfer (CEST) or direct nuclear observation. The dynamic range for MR signal modulation exceeds that of the most optimized gadolinium-based experimental pH-responsive MR imaging probes. In one embodiment, the system is also amenable to further optimization.

It will be appreciated by those skilled in the art that while the disclosed subject matter has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of the following Appendix and of each patent and publication cited therein is hereby incorporated by reference, as if each such patent or publication were individually recited in its entirety.

### Compounds

Provided herein are compounds of Formula (I):

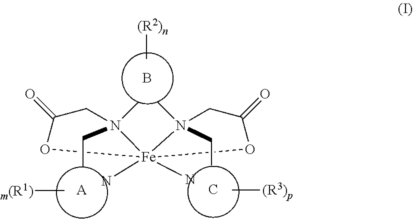

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl; each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein are compounds of Formula (II):

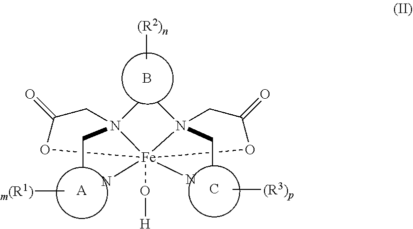

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein is a compound of Formula (III):

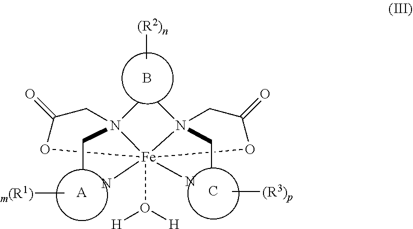

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C_{1 -}6 alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

Also provided herein is a compound of Formula (IV):

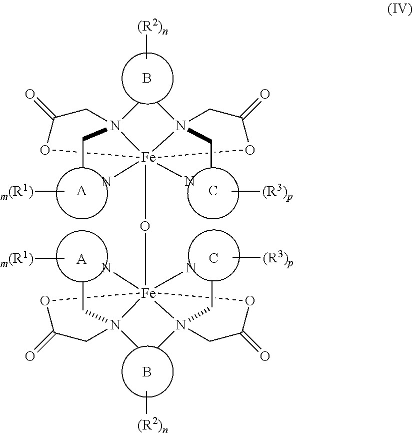

or a pharmaceutically acceptable salt thereof, wherein:

Ring A is a 5-10 membered heteroaryl or a 5-10 membered heterocycloalkyl;

Ring B is phenyl or a C₅₋₇ cycloalkyl;

Ring C is a 5-10 membered heteroaryl or a 5-10 membered heterocycloalkyl;

each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};

m, n, and p are each independently 0, 1, 2, or 3;

each q is independently 0 or 1;

each R^{A} and R^{B} are independently hydrogen or C₁₋₆ alkyl; and

each R^{C} is independently C₁₋₆ alkyl.

In some embodiments, Ring A is 5-10 membered heterocycloalkyl. In some embodiments, Ring A is 5-6 membered heterocycloalkyl. In some embodiments, Ring A is 5 membered heterocycloalkyl. In some embodiments, Ring A is 6 membered heterocycloalkyl.

In some embodiments, Ring A is selected from

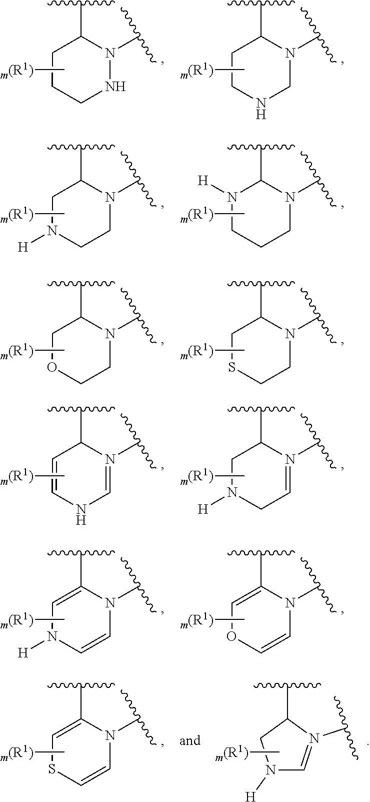

In some embodiments, Ring A is 5-10 membered heteroaryl. In some embodiments, Ring A is 5-6 membered heteroaryl. In some embodiments, Ring A is 5 membered heteroaryl. In some embodiments, Ring A is 6 membered heteroaryl.

In some embodiments, Ring A is selected from

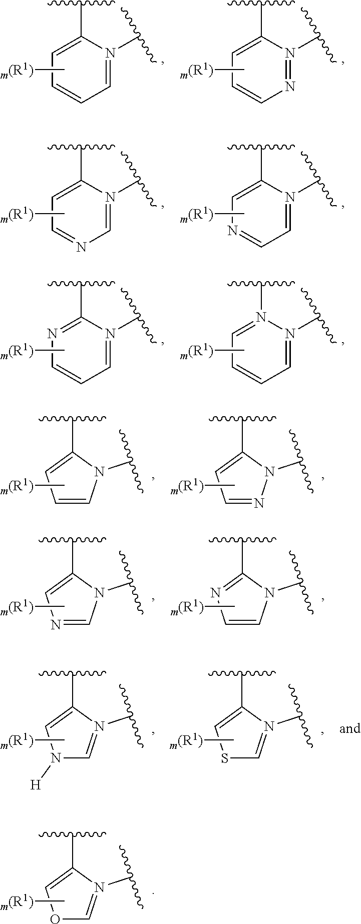

In some embodiments, Ring A is selected from

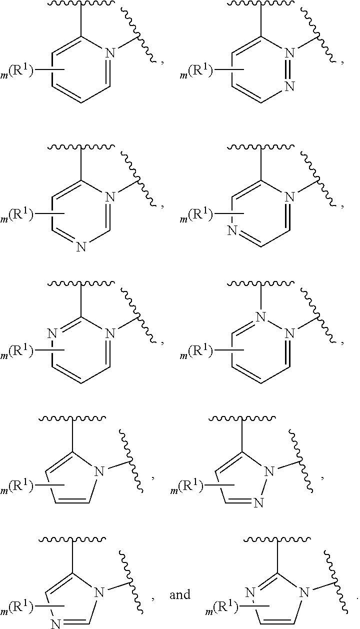

In some embodiments, Ring A is selected from

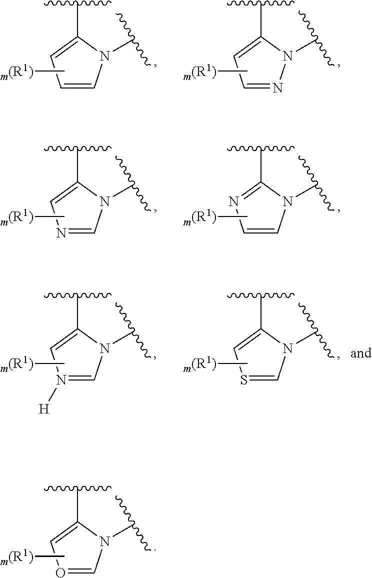

In some embodiments, Ring A is selected from

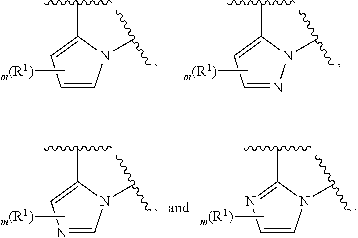

In some embodiments, Ring A is selected from

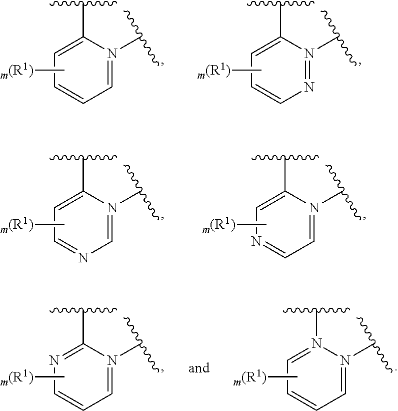

In some embodiments, Ring A is

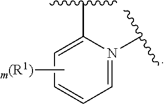

In some embodiments, Ring A is

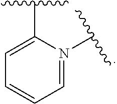

In some embodiments, m is 0. In some embodiments, m is 1, 2, or 3. In some embodiments, m is 1. In some embodiments, m is 2. In some embodiments, m is 3.

In some embodiments, R¹ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, and —(C═O)NHSO₂R^{A}. In some embodiments, R¹ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, and C₃₋₆ cycloalkyl. In some embodiments, R¹ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}C═O)NR^{A}R^{B}, and —(C₁₋₆ alkyl)_{q}NR^{A}(C═O)R^{C}. In some embodiments, R¹ is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

In some embodiments, Ring B is phenyl. In some embodiments, Ring B is C5-7 cycloalkyl. In some embodiments, Ring B is C₆ cycloalkyl.

In some embodiments, n is 0. In some embodiments, n is 1, 2, or 3. In some embodiments, n is 1. In some embodiments, n is 2. In some embodiments, n is 3.

In some embodiments, R² is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q} CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, and —(C═O)NHSO₂R^{A}. In some embodiments, R 2 is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, and C₃₋₆ cycloalkyl. In some embodiments, R² is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, and —(C₁₋₆ alkyl)_{q}NR^{A}(C═O)R^{C}. In some embodiments, R² is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

In some embodiments, Ring C is 5-10 membered heterocycloalkyl. In some embodiments, Ring C is 5-6 membered heterocycloalkyl. In some embodiments, Ring C is 5 membered heterocycloalkyl. In some embodiments, Ring C is 6 membered heterocycloalkyl.

In some embodiments, Ring C is selected from

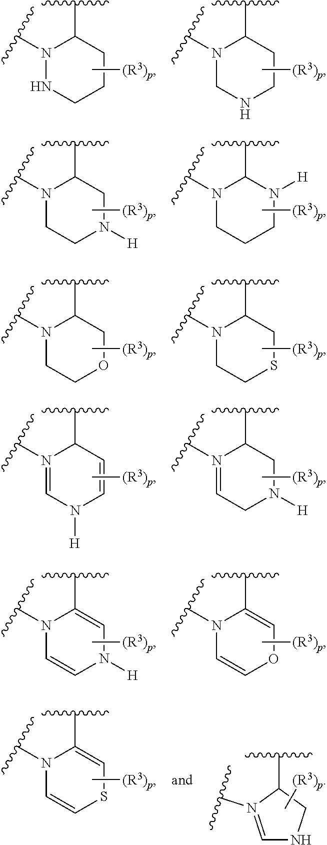

In some embodiments, Ring C is selected from

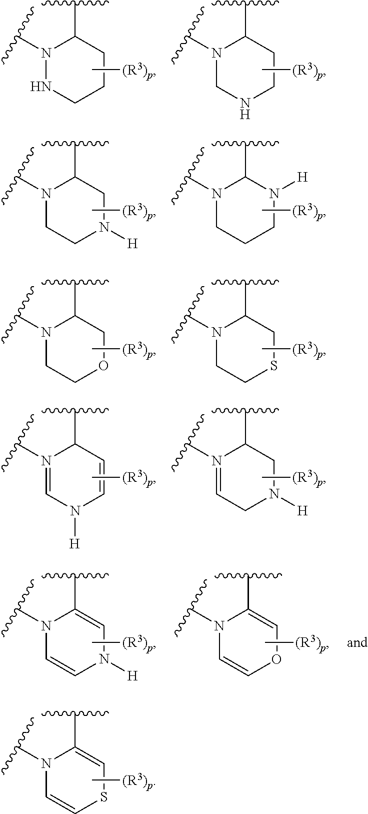

In some embodiments, Ring C is

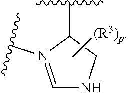

In some embodiments, Ring C is 5-10 membered heteroaryl. In some embodiments, Ring C is 5-6 membered heteroaryl. In some embodiments, Ring C is 5 membered heteroaryl. In some embodiments, Ring C is 6 membered heteroaryl.

In some embodiments, Ring C is selected from

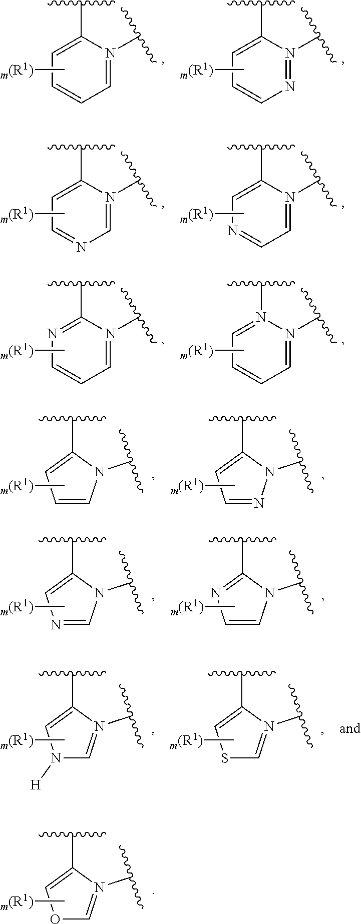

In some embodiments, Ring C is selected from

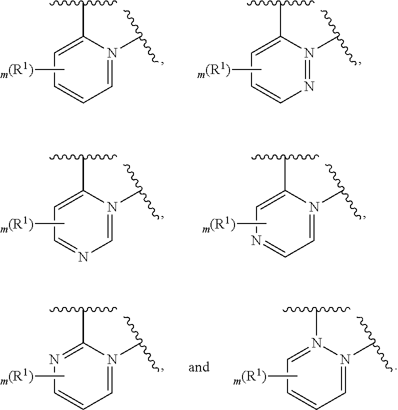

In some embodiments, Ring C is selected from

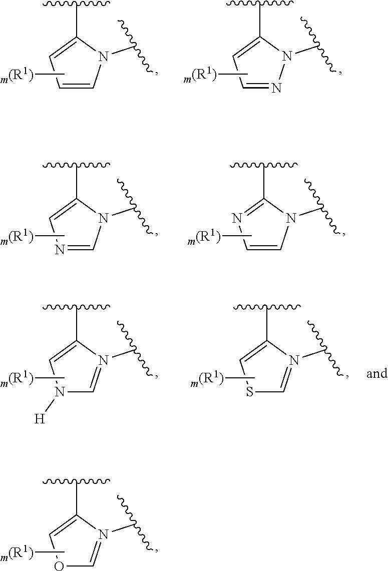

In some embodiments, Ring C is selected from

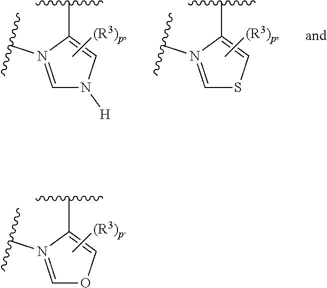

In some embodiments, Ring C is

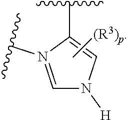

In some embodiments, Ring C is

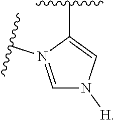

In some embodiment, p is 0. In some embodiments, p is 1, 2, or 3. In some embodiments, p is 1. In some embodiments, p is 2. In some embodiments, p is 3.

In some embodiments, R³ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q} (C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}, NHSO₂R^{A}, and —(C═O)NHSO₂R^{A}. In some embodiments, R³ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, and C₃₋₆ cycloalkyl. In some embodiments, R³ is halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, and —(C₁₋₆ alkyl)_{q}NR^{A}(C═O)R^{C}. In some embodiments, R³ is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

In some embodiments, q is 0. In some embodiments, q is 1.

In some embodiments, R^{A} is hydrogen. In some embodiments, R^{A} is C₁₋₆ alkyl. In some embodiments, R^{A} is C₁₋₄ alkyl. In some embodiments, R^{A} is C₁₋₂ alkyl. In some embodiments, R^{A} is C₁ alkyl.

In some embodiments, R^{B} is hydrogen. In some embodiments, R^{B} is C₁₋₆ alkyl. In some embodiments, R^{B} is C₁₋₄ alkyl. In some embodiments, R^{B} is C₁₋₂ alkyl. In some embodiments, R^{B} is C₁ alkyl.

In some embodiments, R^{C} is C₁₋₆ alkyl. In some embodiments, R^{C} is C₁₋₄ alkyl. In some embodiments, R^{C} is C₁₋₂ alkyl. In some embodiments, R^{C} is C₁ alkyl.

In some embodiments, the compound of Formula (I) is

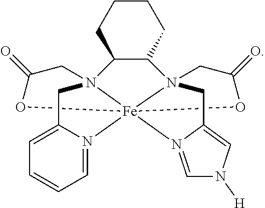

In some embodiments, the compound of Formula (II) is

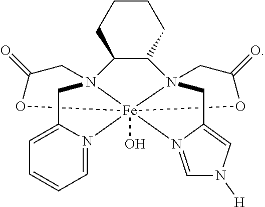

In some embodiments, the compound of Formula (III) is

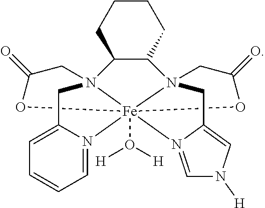

In some embodiments, the compound of Formula (IV) is

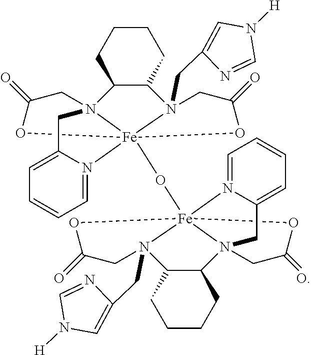

In some embodiments, one of m, n, and p is 1 or 2; and the other two of m, n, and p are 0. In some embodiments, one of m, n, and p is 1; and the other two of m, n, and p are 0. In some embodiments, one of m, n, and p is 2; and the other two of m, n, and p are 0.

In some embodiments, two of R¹, R², and R³ are absent, and the remaining R¹, R², and R³ (i.e., 1, 2, or 3 of that group, depending on m, n, or p) is independently selected from the group consisting of halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl),NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B}. In some embodiments of this paragraph, the remaining R¹, R², and R³ is independently selected from the group consisting of halogen, hydroxyl, cyano, C₁₋₆ alkyl, C₁₋₆ hydroxyalkyl, C₁₋₆ haloalkyl, C₁₋₆ alkoxy, and C₁₋₆ haloalkoxy. In some embodiments of this paragraph, the remaining R¹, R², and R³ is independently selected from the group consisting of C₂₋₆ alkenyl and C₁₋₆ alkynyl. In some embodiments of this paragraph, the remaining R¹, R², and R³ is independently selected from the group consisting of —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, —(C₁₋₆ alkyl)_{q}SO₂R^{A}, and —(C₁₋₆ alkyl)_{q}NHSO₂R^{A}. In some embodiments of this paragraph, the remaining R¹, R², and R³ is independently selected from the group consisting of 4-6 membered heterocycloalkyl, 5-6 membered heteroaryl, and C₃₋₆ cycloalkyl. n some embodiments of this paragraph, the remaining R¹, R², and R³ is independently selected from the group consisting of —(C═O)NHSO₂R^{A}, —P(R^{A})O₂R^{B}, and —PO₃R^{A}R^{B}.

In some embodiments, the compound of Formula (I) has a relaxivity of about 1.0 to about 5.0, for example, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, or any value in between. In some embodiments, the compound of Formula (I) has a relaxivity of about 1.0 to about 3.0. In some embodiments, the compound of Formula (I) has a relaxivity of about 1.0 to about 2.5. In some embodiments, the compound of Formula (I) has a relaxivity of about 1.2 to about 2.3. In some embodiments, the compound of Formula (I) has a relaxivity of about 1.3 to about 2.0. In some embodiments, the compound of Formula (I) has a relaxivity of about 1.4 to about 1.9. In some embodiments, the compound of Formula (I) has a relaxivity of about 1.4 or about 1.9.

In some embodiments, the compound of Formula (II) has a relaxivity of about 0.6 to about 4.5. In some embodiments, the compound of Formula (II) has a relaxivity of about 0.6 to about 4.0. In some embodiments, the compound of Formula (II) has a relaxivity of about 0.6 to about 2.0. In some embodiments, the compound of Formula (II) has a relaxivity of about 0.7 to about 1.9. In some embodiments, the compound of Formula (II) has a relaxivity of about 0.8 to about 1.8. In some embodiments, the compound of Formula (II) has a relaxivity of about 0.9, about 1.4 or about 1.7.

In some embodiments, the compound of Formula (III) has a relaxivity of about 1.0 to about 5.0, for example, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, or any value in between. In some embodiments, the compound of Formula (III) has a relaxivity of about 1.0 to about 3.0. In some embodiments, the compound of Formula (III) has a relaxivity of about 1.0 to about 2.5. In some embodiments, the compound of Formula (III) has a relaxivity of about 1.2 to about 2.3. In some embodiments, the compound of Formula (III) has a relaxivity of about 1.3 to about 2.0. In some embodiments, the compound of Formula (III) has a relaxivity of about 1.4 to about 1.9. In some embodiments, the compound of Formula (III) has a relaxivity of about 1.4 or about 1.9.

In some embodiments, the compound of Formula (IV) has a relaxivity of about 0.02 to about 0.50. In some embodiments, the compound of Formula (IV) has a relaxivity of about 0.03 to about 0.45. In some embodiments, the compound of Formula (IV) has a relaxivity of about 0.05 to about 0.30. In some embodiments, the compound of Formula (IV) has a relaxivity of about 0.06 to about 0.30. In some embodiments, the compound of Formula (IV) has a relaxivity of about 0.06 to about 0.25. In some embodiments, the compound of Formula (IV) has a relaxivity of about about 0.14, or about 0.2.

In some embodiments, the relaxivity of a compound of Formula (I), Formula (II),Formula (III), or Formula (IV) at about pH 6.0 is about 1.0 to about 2.5. In some embodiments, the relaxivity of a compound of Formula (I), Formula (II),Formula (III), or Formula (IV) at about pH 6.0 is about 1.2 to about 2.3. In some embodiments, the relaxivity of a compound of Formula (I), Formula (II), Formula (III), or Formula (IV) at about pH 6.0 is about 1.3 to about 2.0. In some embodiments, the relaxivity of a compound of Formula (I), Formula (II), Formula (III), or Formula (IV) at about pH 6.0 is about 1.4 or about 1.9.

In some embodiments, the relaxivity of a compound of Formula (I), Formula (II), Formula (III), or Formula (IV) at about pH 7.4 is about 0.03 to about 0.50. In some embodiments, the relaxivity of a compound of Formula (I), Formula (II), Formula (III), or Formula (IV) at about pH 7.4 is 0.06 to about 0.25. In some embodiments, the relaxivity of a compound of Formula (I), Formula (II), Formula (III), or Formula (IV) at about pH 7.4 is about 0.07 to about 0.30. In some embodiments, the relaxivity of a compound of Formula (I), Formula (II), Formula (III), or Formula (IV) at about pH 7.4 is about 0.072, about 0.14, or about 0.20.

In some embodiments, the relaxivity of a compound of Formula (I), Formula (II), Formula (III), or Formula (IV) at about pH 6.0 is about 1.2 to about 2.3 and the relaxivity at about pH 7.4 is about 0.06 to about 0.25. In some embodiments, the relaxivity of a compound of Formula (I), Formula (II), Formula (III), or Formula (IV) at pH 6.0 is about 1.2 to about 2.3 and the relaxivity at pH 7.4 is about 0.06 to about 0.25.

The skilled artisan would recognize that the relaxivity values described herein can change based on the field strength of the magnet used in the MR imaging. Such values are also within the scope of the present disclosure.

In some embodiments, the compound is not selected from a compound disclosed in Wang, et al., Inorg. Chem., Vol. 59, No. 23, pp. 17712-17721, which is hereby incorporated by reference in its entirety. In some embodiments, the compound is selected from the compounds described in Examples 1 and 2. In some embodiments, the compound is selected from the compounds described in Tables 1A-1D.

### Pharmaceutical Compositions

In some embodiments, the compounds described herein (e.g., a compound of any one of Formulae (I), (II), (III), (IV), or a mixture of any of the foregoing) are administered alone or as part of a pharmaceutically acceptable composition comprising the compound and a pharmaceutically acceptable carrier. The relative amounts of the compound and the pharmaceutically acceptable carrier will vary depending upon the identity, size, and condition of the subject and further depending upon the route by which the composition is to be administered. In some embodiments, the composition is administered to a subject parenterally, for example, intravenously, intramuscularly, subcutaneously, intracerebrally, or intrathecally. In some embodiments, the composition is administered to a subject intravenously.

In some embodiments, the pharmaceutically acceptable carrier comprises a liquid medium suitable for parenteral administration. In some embodiments, the pharmaceutically acceptable carrier comprises water. In some embodiments, the pharmaceutically acceptable carrier further comprises additional components such as buffers and/or tonicity modifiers.

In some embodiments, the pharmaceutical composition is formulated as a solid to be dissolved in a carrier such as water prior to administration to a subject. In such embodiments, the solid formulation further comprises additional components such as buffers and/or tonicity modifiers.

In some embodiments, the pharmaceutical composition comprises a mixture of the compounds described herein.

### Methods

Some embodiments provide a method for in vivo imaging of a subject comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time. Step (b) comprises obtaining an image of an entire subject (e.g., a full body scan) as well as imaging specific regions of the subject's body. The subject is positioned in a magnet system configured to generate a polarizing magnetic field about at least a portion of a subject. A plurality of gradient coils configured to apply a gradient field to the polarizing magnetic field are energized. A radio frequency (RF) system configured to apply an excitation field to the subject is controlled to acquire magnetic resonance (MR) image data therefrom, and an image of the region of interest is reconstructed from the MR image data.

Some embodiments provide a method for detecting one or more regions of a subject having aberrant pH levels comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time. Regions of the subject having aberrant pH levels that contact the compound result in a modified longitudinal relaxation period of the compound that is reflected in the image.

Some embodiments provide a method of magnetic resonance (MR) imaging a tumor in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method of magnetic resonance (MR) imaging a blood clot in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method of magnetic resonance (MR) imaging a brain lesion in a subject (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method for detecting the presence or absence of a solid tumor in a subject comprising (a) administering to a subject a compound of any one of Formulae (I), (II), (III), (IV), or a mixture of any of the foregoing, or a pharmaceutical composition comprising same; and (b) obtaining a magnetic resonance image of the subject after a period of time.

Some embodiments provide a method for determining the growth rate of a solid tumor in a subject having a solid tumor comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of a disrupted blood-brain-barrier in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of arterial stenosis in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of spinal stenosis in a subject comprising (a) administering to a subject a compound of any one of Formulae (I), (II), (III), (IV), or a mixture of any of the foregoing, or a pharmaceutical composition comprising same; and (b) obtaining a first magnetic resonance image of the subject after a period of time; (c) administering to a subject a compound of any one of Formulae (I), (II), (III), (IV), or a mixture of any of the foregoing, or a pharmaceutical composition comprising same after a second period of time; (d) obtaining a second magnetic resonance image of the subject after a period of time; and (e) comparing the first magnetic resonance image of the subject and the second magnetic resonance image of the subject.

Some embodiments provide a method for in vivo imaging of a subject comprising (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time. Step (b) comprises obtaining an image of an entire subject (e.g., a full body scan) as well as imaging specific regions of the subject's body. The subject is positioned in a magnet system configured to generate a polarizing magnetic field about at least a portion of a subject. A plurality of gradient coils configured to apply a gradient field to the polarizing magnetic field are energized. A radio frequency (RF) system configured to apply an excitation field to the subject is controlled to acquire magnetic resonance (MR) image data therefrom, and an image of the region of interest is reconstructed from the MR image data.

Some embodiments provide a method for detecting one or more regions of a subject having aberrant pH levels comprising ((a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time. Regions of the subject having aberrant pH levels that contact the compound result in a modified longitudinal relaxation period of the compound that is reflected in the image.

Some embodiments provide a method of magnetic resonance (MR) imaging a tumor in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method of magnetic resonance (MR) imaging a blood clot in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method of magnetic resonance (MR) imaging a brain lesion in a subject (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method for detecting the presence or absence of a solid tumor in a subject comprising (a) contacting a cell or region of a subject (e.g., a blood clot, tumor, lesion, or other biological or physiological compartment) with a compound of Formula (III) or a compound of Formula (IV); and (b) obtaining a magnetic resonance image of the subject after a period of time.

Some embodiments provide a method for determining the growth rate of a solid tumor in a subject having a solid tumor comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of a disrupted blood-brain-barrier in a subject (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of arterial stenosis in a subject comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

Some embodiments provide a method for detecting the presence or absence of spinal stenosis in a subject comprising (a) contacting a cell or region of a subject (e.g., a blood clot, tumor, lesion, or other biological or physiological compartment) with a compound of Formula (III) or a compound of Formula (IV); and (b) obtaining a first magnetic resonance image of the subject after a period of time; (c) contacting a cell or region of a subject (e.g., a blood clot, tumor, lesion, or other biological or physiological compartment) with a compound of Formula (III) or a compound of Formula (IV) after a second period of time; (d) obtaining a second magnetic resonance image of the subject after a period of time; and (e) comparing the first magnetic resonance image of the subject and the second magnetic resonance image of the subject.

In some embodiments, the subject is known or suspected to be suffering from a disease or disorder prior to performing the methods described herein, for example, by an approved diagnostic test (e.g., approved by an appropriate regulatory agency such as the USFDA or the EMA), previous imaging studies, displaying particular symptoms, or a combination of any of the foregoing. In some embodiments, the subject has been or is presently undergoing therapy (e.g., standard of care therapy) for the disease or disorder. Accordingly, some embodiments also provide methods of monitoring the progression of a disease or disorder by obtaining multiple images of a subject over time and comparing the images.

In some embodiments, the period of time is about 5 minutes to about 120 minutes, for example, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes, or any value in between. In some embodiments, the period of time is about 5 minutes to about 45 minutes. In some embodiments, the period of time is about 30 minutes to about 60 minutes. In some embodiments, the period of time is about 45 minutes to about 90 minutes. In some embodiments, the period of time is about 60 minutes to about 120 minutes. In some embodiments, the second period of time is about 2 weeks to about 24 months, for example, about 2 weeks, about 4 weeks, about 6 weeks, about 8 weeks, about 10 weeks, about 12 weeks, about 14 weeks, about 16 weeks, about 18 weeks, about 20 weeks, about 22 weeks, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, or any value in between. In some embodiments, the period of time is about 2 weeks to about 3 months. In some embodiments, the period of time is about 2 months to about 6 months. In some embodiments, the period of time is about 4 months to about 12 months. In some embodiments, the period of time is about 8 months to about 18 months. In some embodiments, the period of time is about 12 months to about 24 months.

### Definitions

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted with the indicated groups. The substituents are independently selected, and substitution may be at any chemically accessible position. As used herein, the term “substituted” means that a hydrogen atom is removed and replaced by the indicated substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘalkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘalkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘalkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘalkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “Cₙ₋ₘhydroxyalkyl” refers to an alkyl group substituted with a hydroxy (—OH) group.

As used herein, the term “Cₙ₋ₘalkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “amino” refers to a group of formula —NH₂.

As used herein, the term “aryl”, employed alone or in combination with other terms, refers to an aromatic hydrocarbon group, which may be monocyclic or polycyclic (e.g., having 2, 3, or 4 fused rings). The term “Cₙ₋ₘaryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 14 carbon atoms. In some embodiments, the aryl group has from 5 to 10 carbon atoms. In some embodiments, the aryl group is phenyl or naphthyl. In some embodiments, the aryl group is phenyl.

As used herein, “halo” refers to F, Cl, Br, or I. In some embodiments, a halo is F, Cl, or Br.

As used herein, “Cₙ₋ₘhaloalkoxy” refers to a group of formula —O-haloalkyl having n to m carbon atoms. Example haloalkoxy groups include OCF₃ and OCF₂. An example haloalkoxy group is OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘhaloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CCl₃, CHCl₂, C₂Cl₅ and the like.

As used herein, the term “thio” refers to a group of formula —SH.

As used herein, the term “carbamyl” to a group of formula —C(O)NH₂.

As used herein, the terms “carbonyl” or “oxo”, employed alone or in combination with other terms, refers to a —C(O)- group.

As used herein, “cycloalkyl” refers to non-aromatic cyclic hydrocarbons including cyclized alkyl and alkenyl groups. Cycloalkyl groups can include mono- or polycyclic (e.g., having 2, 3, or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, 12, 13, or 14 ring-forming carbons (i.e., C₃₋₁₄). In some embodiments, the cycloalkyl is a C₃₋₁₄ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C4-10 spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, S, and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-6 monocyclic heteroaryl having 1, 2, or 3 heteroatom ring members independently selected from N, O, S, and B. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, S, and B. In some embodiments, the heteroaryl group contains 3 to 14, 4 to 14, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, isoxazole, thiazole, isothiazole, imidazole, furan, thiophene, triazole, tetrazole, thiadiazole, quinoline, isoquinoline, indole, benzothiophene, benzofuran, benzisoxazole, imidazo[1,2-b]thiazole, purine, triazine, thieno[3,2-b]pyridine, imidazo[1,2-a]pyridine, 1,5-naphthyridine, 1H-pyrazolo [4,3-b]pyridine, and the like.

A five-membered heteroaryl is a heteroaryl group having five ring-forming atoms wherein one or more (e.g., 1, 2, or 3) of the ring-forming atoms are independently selected from N, O, B, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine.

A six-membered heteroaryl ring is a heteroaryl with a ring having six ring-forming atoms wherein one or more (e.g., 1, 2, or 3) ring atoms are independently selected from N, O, S, and B. Exemplary six-membered ring heteroaryls are pyridyl, pyrazinyl, pyrimidinyl, triazinyl and pyridazinyl.

As used herein, “heterocycloalkyl” refers to monocyclic or polycyclic heterocycles having at least one non-aromatic ring (saturated or partially saturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfide (e.g., C(O), S(O), C(S), or S(O)₂, etc). Heterocycloalkyl groups include monocyclic and polycyclic (e.g., having 2, 3, or 4 fused rings) systems. Included in heterocycloalkyl are monocyclic and polycyclic 3-14-, 4-14-, 3-10-, 4-5-10-, 4-7-, 5-7-, 5-6-, 5- or 6- membered heterocycloalkyl groups. Heterocycloalkyl groups can also include spirocycles and bridged rings (e.g., a 5-14 membered bridged biheterocycloalkyl ring having one or more ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds, i.e., is partially saturated. In some embodiments, the heterocycloalkyl group contains 0 to 2 double bonds.

Example heterocycloalkyl groups include pyrrolidonyl, pyrrolidin-2-one, 1,3-isoxazolidin-2-one, pyranyl, tetrahydropyran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3 ]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl and the like. In some embodiments, the heterocycloalkyl group is pyrrolidonyl, pyrrolidin-2-one, 1,3-isoxazolidin-2-one, pyranyl, tetrahydropuran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, or azepanyl.

In some embodiments, the heterocycloalkyl group contains 3 to 14 ring-forming atoms, 4 to 14 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S, and B and having one or more oxidized ring members. In some embodiments, the heterocycloalkyl is a monocyclic or bicyclic 4-10 membered heterocycloalkyl having 1, 2, 3, or 4 heteroatoms independently selected from N, O, S, and B and having one or more oxidized ring members.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group. “Oxo” can also refer to an oxygen atom as a ligand to a metal atom, such as an iron atom.

The term “relaxivity” as used herein, refers to the increase in either of the MR quantities 1/T₁ or 1/T₂ per millimolar (mM) concentration of paramagnetic ion or contrast agent, which quantities may be different if the contrast agent contains a multiplicity of paramagnetic ions, wherein T₁ is the longitudinal or spin-lattice relaxation time, and T₂ is the transverse or spin-spin relaxation time of water protons or other imaging or spectroscopic nuclei, including protons found in molecules other than water. Relaxivity is expressed in units of mM⁻¹s⁻¹.

“Subject” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the subject is a human.

The term “about” as used in connection with a numerical value throughout the specification and the claims denotes an interval of accuracy, familiar and acceptable to a person skilled in the art. Such interval of accuracy is, for example, ±10%.

### EXAMPLES

The following examples are illustrative and not intended to be limiting.

### Abbreviations

| | |
|---|---|
| H₂PyCy2AI | 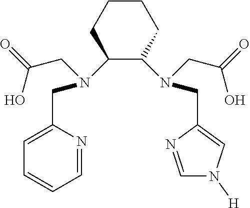 |
| H₂PyCy2AI•2TFA | Isolated for of H₂PyC2AI. The compound |
| | was isolated as an adduct with 2 |
| | trifluoroacetic acid molecules. |
| PyCy2AI | The double deprotonated form of H₂PyCy2AI |
| Fe—PyCy2AI | Generic term encompassing all species |
| | potentially present in pH 6-7.4 aqueous |
| | solution comprised of 1:1 Fe and H₂PyCy2AI |
| [Fe(PyCy2AI)]⁺ | 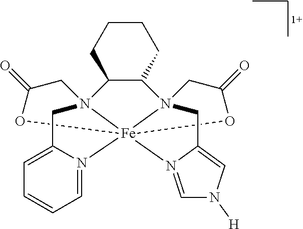 |
| [Fe(PyCy2AI)(H₂O)]⁺ | 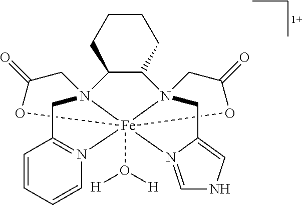 |
| [Fe(PyCy2AI)(OH)] | 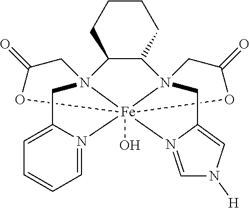 |
| [(Fe—PyCy2AI)₂O] | 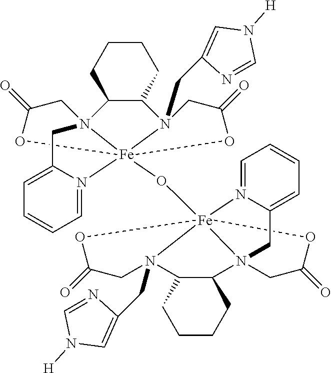 |
| H2PyCy2AI—Me | 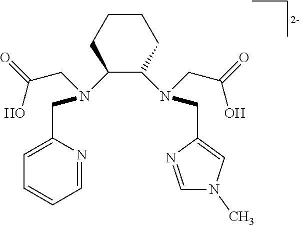 |
| Fe—PyCy2AI—Me | Generic term encompassing all species |
| | potentially present in pH 6-7.4 aqueous |
| | solution comprised of 1:1 Fe and |
| | H₂PyCy2AI—Me |
| [Fe—(PyCy2AI—Me)]⁺ | 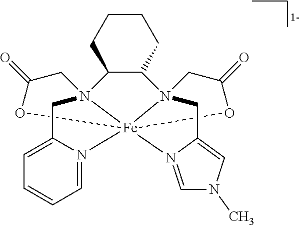 |
| [Fe—PyCy₂AI—Me)(H₂O)]⁺ | 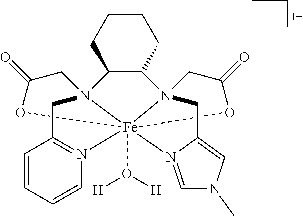 |
| [Fe—PyCy₂AI—Me)(OH)] | 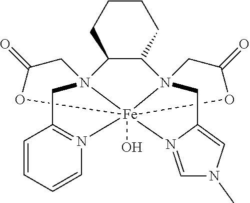 |
| [(Fe—PyCy2AI)₂O] | 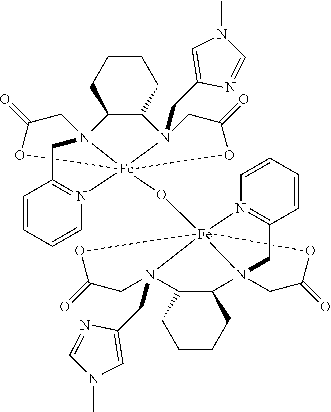 |
| EDTA | Ethylenediaminetetraacetic acid |
| HEDTA | Hydroxyethylethylenediaminetriacetic acid |
| Fe-EDTA | 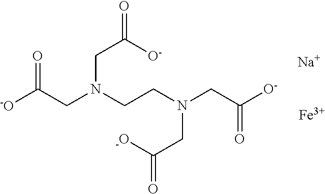 |
| [Fe(HEDTA)] | 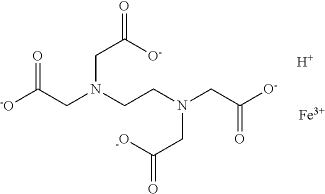 |
| CyDTA | 1,2-Cyclohexylenedinitrilotetraacetic acid |
| PDTA | N,N,N′,N′-1,2-propylenediaminetetracetic |
| | acid |
| PyC3A | 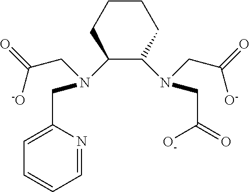 |

### Methods

General. All chemicals and solvents were purchased commercially and used without further purification.

High-performance Liquid Chromatography (HPLC) and Liquid chromatography-mass spectrometry (LC-MS) methods. HPLC and LC-MS were performed using an Agilent 1260 Series apparatus with an LC/MSD trap and Daly conversion dynode detector with UV detection at 220, 254, and 280 nm or an Agilent 1260 apparatus interfaced to an Agilent 8800-QQQ ICP-MS for Mn detection.

The method used for purification are as follows:

(P1) Phenomenex C18 column (250×21.8 cm); eluent A: H₂O/0.1% trifluoroacetic acid (TFA), eluent B: MeCN/0.1% TFA; gradient: starting from 95% A/5% B held for 8 min, the fraction of B increased to 50% over 10 min, 50% to 95% B over 2 min. The column was washed with 95% B for 3 min and then ramped to 5% B in 2 min. The system was re-equilibrated at 5% B for 3 min; flow rate 15 mL/min.

(P2) eluent C: 50 mM ammonium acetate buffer, pH 6.5; eluent D: 10% 50 mM ammonium acetate buffer, pH 6.5 and 90% MeCN; gradient: starting from 95% A/5% B held for 8 min, the fraction of B increased to 50% over 10 min, 50% to 95% B over 2 min. The column was washed with 95% B for 3 min and then ramped to 5% B in 2 min. The system was re-equilibrated at 5% B for 3 min; flow rate 15 mL/min.

(A1) Waters X-bridge C18 column (150 □4.6 mm); eluent A: H2O/0.1% formic acid, eluent B: MeCN/0.1% formic acid; gradient: 5% B for 2 min, 5% B to 50% B over 7 min, 50% B to 95% B over 1 min, 95% B for 2 min, 95% B to 5% B for 1 min, then 5% B for 2 min; flow rate 0.7 mL/min.

(A2) Luna C18 column (100 mm×2 mm 100 Å); eluent C, 10 mM ammonium acetate in water; eluent D, 90% MeCN and 10% 10 mM ammonium acetate in water; gradient, 5% D for 1 min, 5% D to 95% D in 10 min, 95% D for 2 min, 95 to 5% D in 1 min, 5% D for 1 min; flow rate at 0.7 mL/min.

Metal ion quantification. Fe complex concentration in solution samples were quantified using an Agilent 8800-QQQ ICP-MS system. Samples with diluted with 0.1% Triton X-100 in 5% nitric acid. A linear calibration curve for metal ranging from 1.0 ppb to 1000 ppb Fe was generated daily for the quantification.

NMR. NMR spectra were recorded on a 500 MHz Joel spectrometer. Chemical shifts are reported in δ (ppm). For ¹H and ¹³C NMR spectra, the residual solvent peaks were used as internal reference except for ¹³C NMR recorded in D₂₀ where 0.05 wt. % 3-(trimethylsilyl)propionic-2,2,3,3-d₄ acid, sodium salt was used as the internal reference.

pH-potentiometric measurements: pH-potentiometric measurements were performed using an Orion ROSS Ultra pH electrode and temperature-controlled reaction vessel held at 310 K. A standardized solution of 0.10 M NaOH was used as the titrant. The electrode was calibrated prior to each titration by titrating a standardized HCl(aq) solution at ionic strength 0.10 using NaCl as the inert electrolyte with the standardized NaOH titrant. A working slope and intercept were generated by plotting mV as a function of calculated pH, which enabled direct conversion of electrode readings to [H⁺] during sample titrations. pH values recorded during the titrations refers to hydrogen ion concentration. All titration samples were prepared in solutions of 0.10 M NaCl in distilled, de-ionized water. Ligand solutions were prepared by dissolving a weighed quantity into the electrolyte and concentration was calculated from the effective weight of the ligand. Moles ligand present in titrand was also confirmed by measuring the mol added NaOH required to span each inflection point. Solutions of 1:1 ligand: Fe were prepared by adding an appropriate volume of standardized FeCl₃ in 0.088 M HCl solution to a weighed quantity of ligand, the solutions were then adjusted with water and 1 M NaCl to ionic strength 0.10. The data was analyzed using the Hyperquad2013 software package.

### Example 1.[Fe-HPyCy2AI](TFA)₂

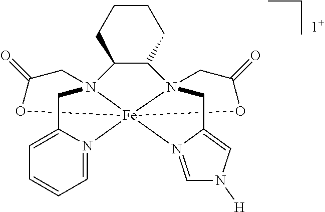

Step 1: (±)-trans-N-(1H-imidazol-4-yl)methyl)-N′-(picolyl)cyclohexane-1,2-diamine

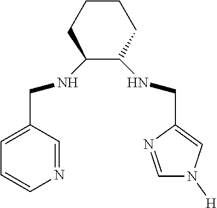

To a stirring solution of (±)-trans-N-(picolyl)-1,2-diaminocyclohexane (0.50 g, 2.44 mmol, 1 equiv.) in methanol was slowly added 4-imidazolecarboxaldehyde (0.29 g, 3.04 mmol, 1.25 equiv.). The mixture was stirred for 2 hours at room temperature followed by concentrating to dryness. The mixture was dissolved in 50 mL of dichloromethane and added to a stirring solution of sodium borohydride (0.46 g, 12.20 mmol, 5 equiv.) in 150 mL methanol over a period of 30 min. The mixture was stirred for 2 hours followed by addition of distilled water. The solvent was removed under reduced pressure leading to a white solid. The solid was then dissolved in water. The pH of the resulting mixture was adjusted to pH>11 by addition of 1 M NaOH, and then the mixture was extracted three times with dichloromethane. The combined organic layer was evaporated under reduced pressure resulting in a colorless oil. The oil was dissolved in water, and the pH was adjusted to pH 6.5 with 6 M HCl. The oil was purified by preparative RP-HPLC using the C18 column and method P2. RP-HPLC fractions containing pure product were freeze dried to yield (±)-trans-N-((¹H-imidazol-4-yl)methyl)-N′-(picolyl)cyclohexane-1,2-diamine as a white solid (0.50 g, 1.76 mmol, 76%). ¹H NMR (500 MHZ, CD₃OD): δ 8.35 (t, J=4.3 Hz, 1H), 7.76 (m, 2H), 7.44-7.37 (t, J=6.7 Hz, 1H), 7.25 (m, 2H), 4.21 (dd, J=14.0, 5.3 Hz, 1H), 4.12 (dd, J=15.1, 5.3 Hz, 1H), 4.04 (dd, J=14.0, 4.8 Hz, 1H), 3.94 (dd, J=15.4, 5.3 Hz, 1H), 2.73 (m, 1H), 2.50 (m, 1H), 2.22 (d, J=12.8 Hz, 2H), 1.78 (m, 2H), 1.27 (m, 2H), ¹³C NMR (125.7 MHZ, CD₃OD): δ 177.95 (acetate), 157.93, 148.34, 137.46, 136.10, 131.87, 122.78, 122.60, 118.15, 60.24, 59.12, 49.47, 41.36, 30.07, 28.18, 24.37, 24.05, 22.28 (acetate). ESI-MS: m/z=286.2 [M+H]⁺; ESI-MS calculated for [C₁₆H₂₃N₅+H]⁺: m/z=286.2.

Step 2: (±)-trans-N-((1H-imidazol-4-yl)methyl)-N′-(picolyl)-N,N′-cyclohexane-1,2-diamine diacetate bis-trifluoroacetic acid, (H₂PyCy2AI*2TFA)

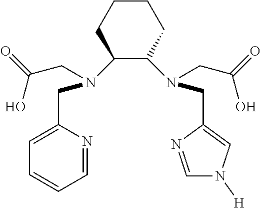

To pure (±)-trans-N-((1H-imidazol-4-yl)methyl)-N′-(picolyl)cyclohexane-1,2-diamine (0.27 g, 0.95 mmol, 1 equiv.) in 10 mL MeOH was added NaHCO₃ (0.24 g, 3.78 mmol, 4 equiv.) and glyoxylic acid monohydrate (0.35 g, 3.78 mmol, 4 equiv.). A batch of sodium cyanoborohydride (0.32 g, 3.78 mmol, 4 equiv.) was added portionwise over the course of 8 hours. After stirring for 16 hours at room temperature, the reaction mixture was filtered and purified by RP-HPLC using preparative method P1. RP-HPLC fractions containing pure product were freeze dried to yield (±)-trans-N-((1H-imidazol-4-yl)methyl)-N′-(picolyl)-N,N′-cyclohexane-1,2-diamine diacetate bis-trifluoroacetic acid as a white solid (0.24 g, 0.38 mmol, 40%). pH-potentiometric analysis of (±)-trans-N-((1H-imidazol-4-yl)methyl)-M-(picolyl)-N,N′-cyclohexane-1,2-diamine diacetate bis-trifluoroacetic acid in the presence and absence of Fe³⁺ were consistent with four ionizable protons per mol ligand. ¹H NMR (500 MHZ, CD₃OD with 0.5 wt. % NaOD): δ 8.79 (s, 1H), 8.53 (s, 1H), 7.97 (s, 1H), 7.68 - 7.43 (m, 3H), 4.45 (m, 2H), 4.13 (d, J=13.9 Hz, 1H), 4.08-3.79 (m, 3H), 3.63 - 3.34 (m, 3H), 3.23 (d, J=10.6 Hz, 1H), 2.22 (d, J=41.9 Hz, 2H), 1.96-1.77 (m, 2H), 1.59 (d, J=10.7 Hz, 1H), 1.50-1.31 (m, 3H). ¹³C NMR (125.7 MHZ, CD₃OD with 0.5 wt. % NaOD): δ 176.2, 171.0, 162.2 (TFA), 161.9 (TFA), 161.6 (TFA), 161.4 (TFA), 156.6, 149.0, 137.7, 136.6, 130.0, 124.9, 123.0, 120.4 (TFA), 120.1, 118.1 (TFA), 115.7 (TFA), 113.4 (TFA), 60.5, 58.7, 58.0, 51.3, 50.7, 49.3, 49.0, 24.3, 23.9, 23.4. ESI-MS: m/z=402.1 [M+H]⁺; ESI-MS calculated for [C₂₀H₂₇N₅O₄+H]⁺: m/z=402.2.

### Step 3: [Fe-PyCy2AI](TFA)₂

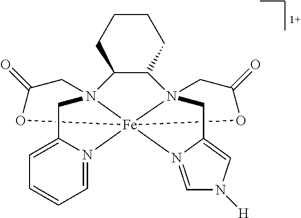

FeCl₃ (23 mg, 0.14 mmol) was added to a 4 ml water solution of (±)-trans-N-((1H-imidazol -4-yl)methyl)-N′-(picolyl)-N,N′-cyclohexane-1,2-diamine diacetate bis-trifluoroacetic acid (58 mg, 0.092 mmol). The pH was adjusted to 3.5 with 3 M NaOH. Complex formation was monitored by LC-MS. The crude product was filtered and subjected to preparative HPLC (method P1) purification. [Fe-HPyCy2AI](TFA)₂ (38 mg, 0.056 mmol and 60%) was isolated as pure product. ESI-MS: m/z=455.0 [M+H]⁺; ESI-MS calculated for [C₂₀H₂₅FeN₅O₄]⁺:455.1. Elemental analysis calculated for C₂₀H₂₅N₅FeO₄1.92TFA*1.20H₂O: C, 41.15; H, 4.24; N, 10.06. Found: C, 41.17; H, 4.24; N, 10.06.

### Example 2. [Fe-(PyCy2AI-Me)](TFA)₂

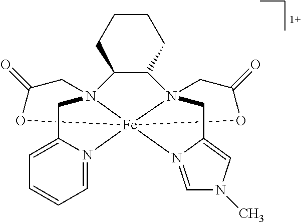

Step 1: (±)-trans-N-((1-methyl-1H-imidazol-4-yl)methyl)-N′-(picolyl)cyclohexane-1,2-diamine bis-trifluoroacetic

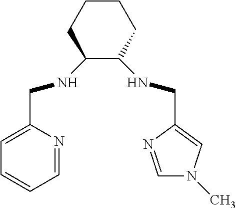

To a stirring solution of (±)-trans-N-(picolyl)-1,2-diaminocyclohexane (0.50 g, 2.44 mmol, 1 equiv.) in methanol was added 1-methyl-1H-imidazole-4-carbaldehyde (0.34 g, 3.04 mmol, 1.25 equiv.) slowly. The mixture was stirred for 2 hours at room temperature followed by concentrating to dryness. The mixture was dissolved in 50 mL of dichloromethane and added to a stirring solution of sodium borohydride (0.46 g, 12.20 mmol, 5 equiv.) in 150 mL methanol over a period of 30 min. The mixture was stirred for 2 hours followed by addition of distilled water. The solvent was removed under reduced pressure leading to a white solid. The solid was then dissolved in water. The pH of the resulting mixture was adjusted to pH>11 by addition of 1 M NaOH. The mixture was extracted three times with dichloromethane. The combined organic layer was evaporated under reduced pressure resulting in colorless oil. The oil was dissolved in water. The pH of the resulting solution was adjusted to pH 6.5 with 6M HCl, and the solution was purified by preparative RP-HPLC using the C18 column and method P1. RP-HPLC fractions containing pure product were freeze dried to yield (±)-trans-N-((1-methyl-1H-imidazol-4-yl)methyl)-N′-(picolyl)cyclohexane-1,2-diamine bis-trifluoroacetic as a white solid. (0.60 g, 1.45 mmol, 60%). ¹H NMR (500 MHZ, CD₃OD): δ 8.77 (s, 1H), 8.60 (d, J=5.0 Hz, 1H), 7.96 (td, J=7.7, 1.9 Hz, 1H), 7.55 (d, J=7.7 Hz, 1H), 7.52 (s, 1H), 7.48 (dd, J=7.7, 4.9 Hz, 1H), 4.47 (d, J=15.2 Hz, 1H), 4.33 (d, J=15.2 Hz, 1H), 4.17 (d, J=14.6 Hz, 1H), 3.90 (d, J=18.9 Hz, 3H), 2.89 (td, J=11.2, 4.2 Hz, 1H), 2.71 (td, J=10.9, 4.0 Hz, 1H), 2.29 (t, J=13.5 Hz, 2H), 1.83 (t, J=10.3 Hz, 2H), 1.42 (qd, J=12.3, 4.0 Hz, 1H), 1.38 - 1.28 (m, 1H), 1.27 - 1.12 (m, 1H). 13 C NMR (125.7 MHZ, CD₃OD): δ 161.71 (TFA), 161.43 (TFA), 161.15 (TFA), 160.87 (TFA), 151.95, 147.83, 139.05, 135.91, 132.28, 124.06, 123.51, 121.15, 120.12 (TFA), 117.79 (TFA), 115.47 (TFA), 113.14 (TFA), 60.25, 58.25, 47.01, 39.16, 34.75, 29.44, 27.59, 24.01, 23.82. ESI-MS: m/z=300.1 [M+H]⁺; ESI-MS calculated for [C₁₇H₂₅N₅+H]⁺: m/z=300.2

Step 2: (±)-trans-N-((1-methyl-1H-imidazol-4-yl)methyl)-N′-(picolyl)-N,N′-cyclohexane-1,2-diamine diacetate bis-trifluoroacetic, (H₂PyCy2AI-Me)

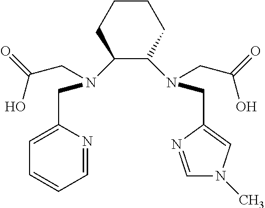

To pure (±)-trans-N-((1-methyl-1H-imidazol-4-yl)methyl)-N′-(picolyl)cyclohexane-1,2-diamine (0.29 g, 0.69 mmol, 1 equiv.) in 10 mL MeOH was added NaHCO₃ (0.17 g, 2.76 mmol, 4 equiv.) and glyoxylic acid monohydrate (0.25 g, 2.76 mmol, 4 equiv.). A batch of sodium cyanoborohydride (0.23 g, 2.76 mmol, 4 equiv.) was added portion wise over the course of 8 hours. After stirring for 16 hours at room temperature, the reaction mixture was filtered and purified by RP-HPLC using preparative method P1. Preparative RP-HPLC fractions containing pure product were freeze dried to yield (±)-trans-N-((1-methyl-1H-imidazol-4-yl)methyl)-N′-(picolyl)-N,N′-cyclohexane-1,2-diamine diacetate bis-trifluoroacetic as a white solid (0.30 g, 0.57 mmol, 83%). ¹H NMR (500 MHZ, CD₃OD with 0.5 wt. % NaOD): δ 8.51 (m, 2H), 7.87 (t, J=8.0 Hz, 1H), 7.61 (s, 1H), 7.48 (s, 1H), 7.42 (s, 1H), 4.36 (s, 2H), 4.11 (d, J=14.0 Hz, 2H), 3.88 (m, 4H), 3.74-3.34 (m, 4H), 3.34-3.06 (m, 2H), 2.40-2.05 (m, 2H), 1.93-1.81 (m, 2H), 1.62-1.26 (m, 2H). ¹³C NMR (125.7 MHZ, CD₃OD with 0.5 wt. % NaOD): δ 172.87, 169.54, 161.81 (TFA), 161.53 (TFA), 161.25 (TFA), 160.97 (TFA), 150.74, 148.09, 139.05, 136.14, 130.90, 124.83, 124.46, 122.48, 120.29 (TFA), 117.95 (TFA), 115.63 (TFA), 113.30 (TFA), 64.09, 61.53, 60.60, 55.03, 51.35, 46.72, 34.84, 24.34, 24.16, 23.96. ESI-MS: m/z=416.1 [M+H]⁺; ESI-MS calculated for [C₂₁H₂₉N₅O₄+H]⁺: m/z=416.2.

Step 3: [Fe-(PyCy2AI-Me)](TFA)₂

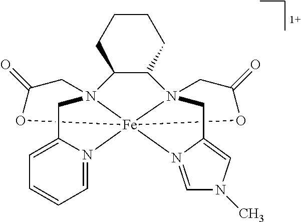

FeCl₃ (61 mg, 0.76 mmol) was added to a 4 ml water solution of (±)-trans-N-((1-methyl -1H-imidazol-4-yl)methyl)-N′-(picolyl)-N,N′-cyclohexane-1,2-diamine diacetate bis-trifluoroacetic (200 mg, 0.31 mmol, assuming bis-TFA adduct). The pH was adjusted to 3.5 by adding 3 M NaOH. Complex formation was monitored by LC-MS. The crude product was filtered and subjected to preparative HPLC (method P1) purification. [Fe-(PyCy2AI-Me)](TFA)₂ was isolated (180 mg, 0.26 mmol, 83%, assuming product isolated as bis-TFA adduct) as a pale yellow solid. ESI-MS: m/z=469.0 [M+H]⁺; ESI-MS calculated for [C₂₁H₂₇FeN₅O₄]⁺:469.1. Elemental analysis calculated for C₂₁H₂₇N₅FeO₄*2.52TFA*0.72H₂O*0.0.70MeCN: C, 40.89; H, 4.18; N, 9.99. Found: C, 40.78; H, 4.17; N, 10.00.

### Example 3. Characterization Data

Liquid chromatography-mass spectrometry (LC-MS)

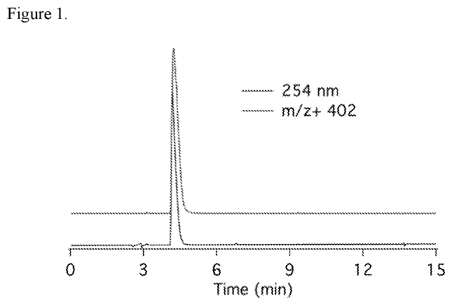

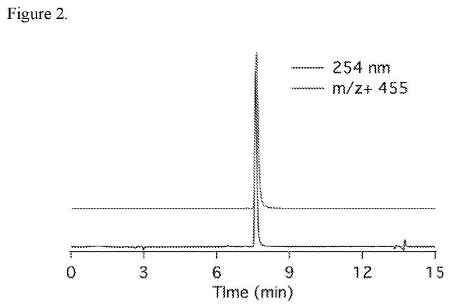

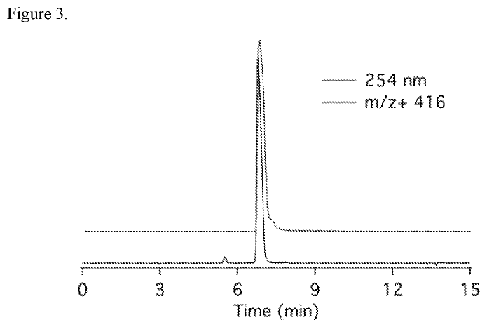

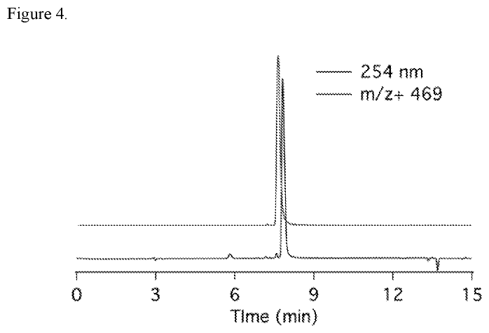

FIG. 1, FIG. 2, FIG. 3, and FIG. 4 show LC and MS traces of PyCy2AI, Fe-PyCy2AI-Me, PyCy2AI-Me, and PyCy2AI-Me, respectively. The LC-MS data were recorded using method A1.

### High-performance Liquid Chromatograph (HPLC)

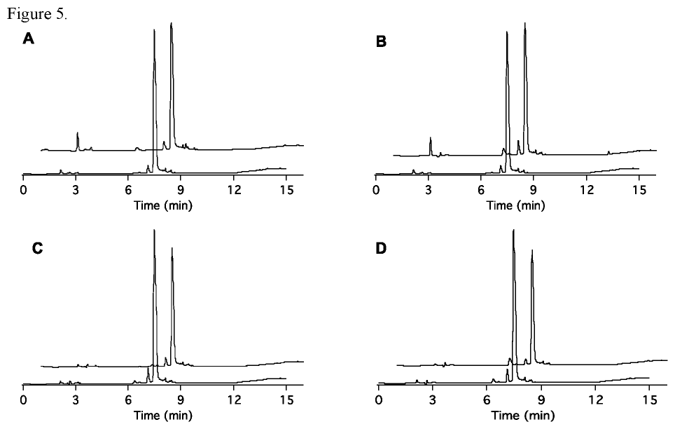

FIG. 5 shows HPLC traces of Fe-PyCy2AI at 254 nm detection recorded within 30 min (front trace) and 24 hours after (offset trace) of dissolution in buffered solutions of A: pH 4.2 (100 mM phosphate), B: pH 7.4 (100 mM phosphate), C: pH 9.0 (100 mM borate), and D: pH 11.2 (100 mM borate). The HPLC data were recorded using method A1.

UV-vis spectroscopy

UV-vis spectra were recorded on a SpectraMax M2 spectrophotometer using quartz cuvettes with a 1 cm path length. Extinction coefficients recorded at pH 9 (ε, M⁻¹cm⁻¹) were estimated by dividing absorbance by sample Fe concentration.

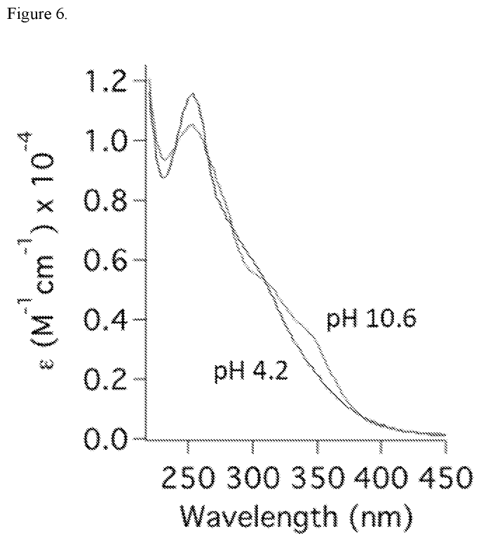

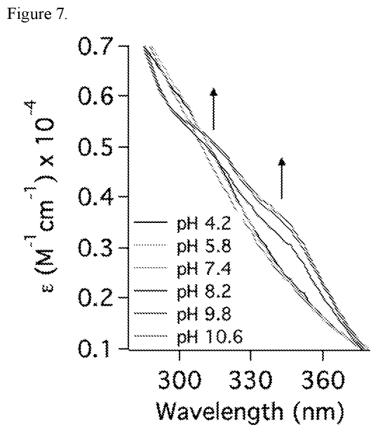

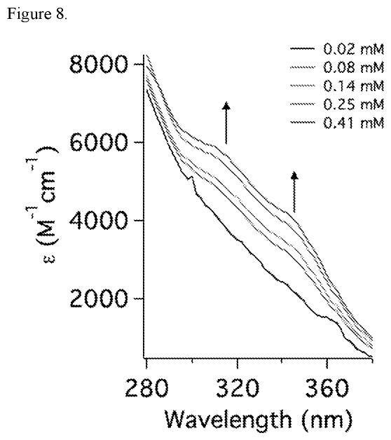

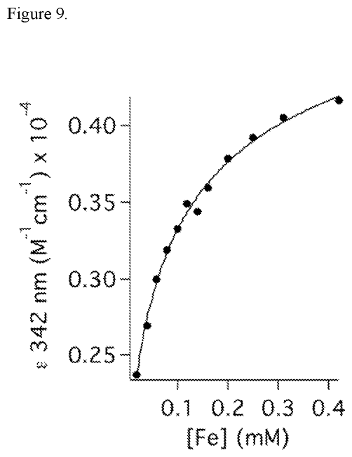

FIG. 6 shows UV-vis spectra of Fe-PyCy2AI recorded at pH 4.2 and pH 10.6 at 310 K. FIG. 7 shows pH dependence on UV-vis absorbance of 0.1 mM of Fe-PyCy2AI between 280-380 nm at 310 K. FIG. 8 shows UV-vis absorbance of different concentrations of Fe-PyCy2A between 280 and 380 nm at pH 9.0 (100 mM borate buffer) and 310 K. FIG. 9 shows the absorbance at 342 nm of different concentrations of Fe-PyCy2AI at pH 9.0 (100 mM borate buffer) and 310 K.

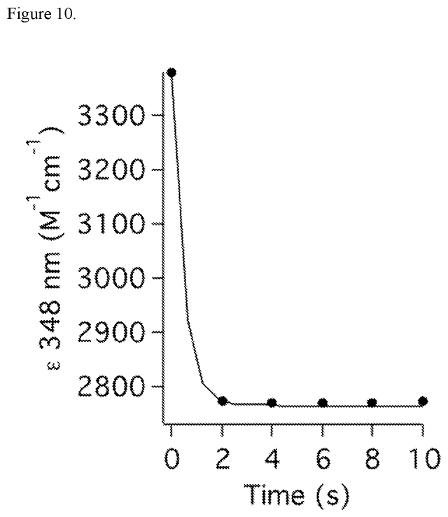

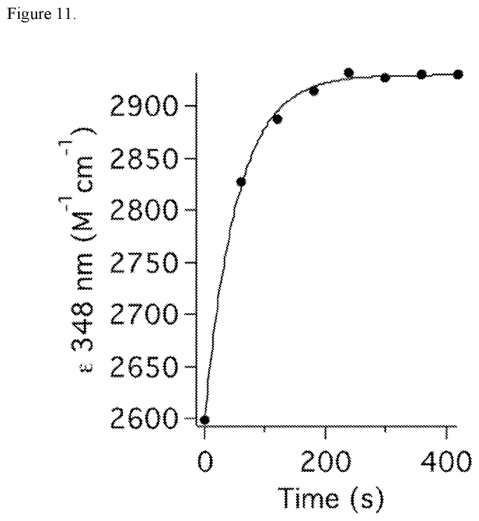

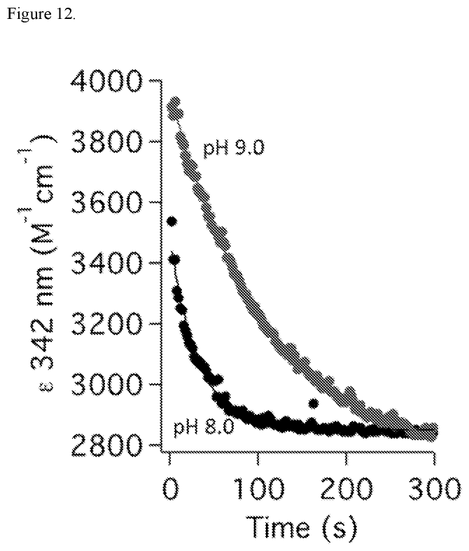

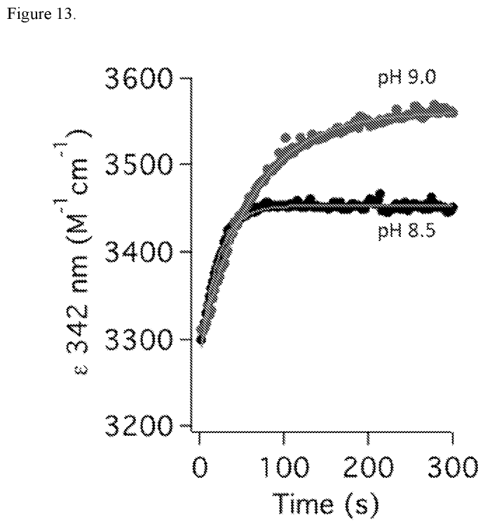

FIG. 10 shows change in UV-vis absorbance at 348 nm after diluting a 5.0 mM aliquot of pH 7.4 solution of Fe-PyCy2AI to 0.5 mM in pH 5 buffer (100 mM phosphate) at 310 K. FIG. 11 shows change in UV-vis absorbance at 348 nm after diluting 5.0 mM aliquot of pH 5.0 solution of 5.0 mM Fe-PyCy2AI to 0.5 mM in pH 9.0 buffer (100 mM borate) at 310 K. FIG. 12 shows change in UV-vis absorbance at 348 nm after diluting a 5.0 mM aliquot of pH 9.0 Fe-PyCy2AI solution to 0.1 mM in pH 8.0 or pH 9.0 buffered solutions (100 mM borate) at 310 K. FIG. 13 shows change in UV-vis absorbance at 348 nm after adding a 5.0 mM aliquot of pH 5.0 solution of 0.1 mM Fe-PyCy2AI to a pH 8.5 or pH 9.0 buffered solution (100 mM borate) at 310 K.

pH titration

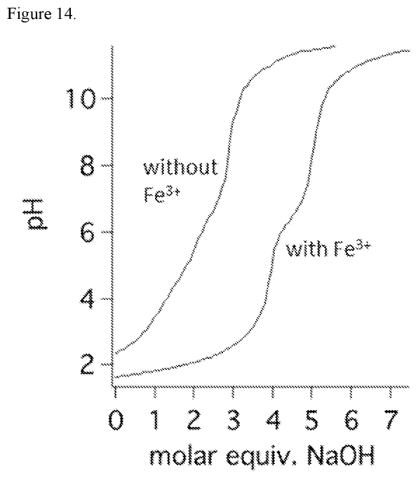

FIG. 14. Shows pH titration curves of 7.0 mM H₂PyCy2AI*2TFA in the presence and absence of 1 molar equivalent Fe³⁺ at 310 K, I=0.1 M NaCl.

Electron paramagnetic resonance spectroscopy (EPR)

Solutions of 1 mM Fe³⁺-PyC2A4Imd were prepared by dissolving the lyophilized powder in 100 mM phosphate buffer (pH 4.2-7.8 samples) or 100 mM borate (pH 8.2-9.8 samples) at the desired pH. The solutions were then transferred into quartz EPR tubes (Wilmad Lab-Glass 727-SQ-250M), and frozen using liquid nitrogen. CW X-band EPR spectra were collected at indicated temperatures and powers at the Analytical Spectroscopy Lab (Ohio State University) using a Bruker EMXPlus instrument equipped with a Cold Edge cryogen-free helium cryostat and recirculation system and an Oxford Instruments MercurylTC temperature controller. All presented spectra were obtained under non-saturating conditions using a microwave frequency of 9.37 MHz and a modulation frequency and amplitude of 100 kHz and 10 G, respectively. EPR spectral simulations were performed using the EasySpin (version 5.2.28) toolbox within MATLAB.10. Background signals were removed from the spectra using a spline subtraction in IGOR Pro 8.04 (Wavemetrics, Lake Oswego, OR) prior to simulation.

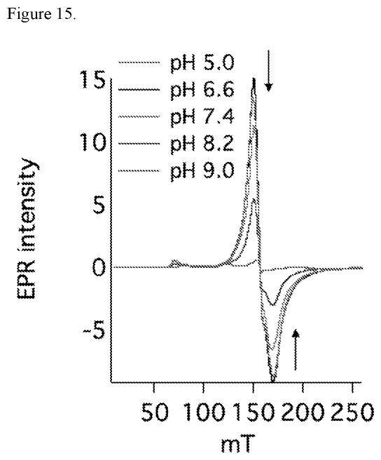

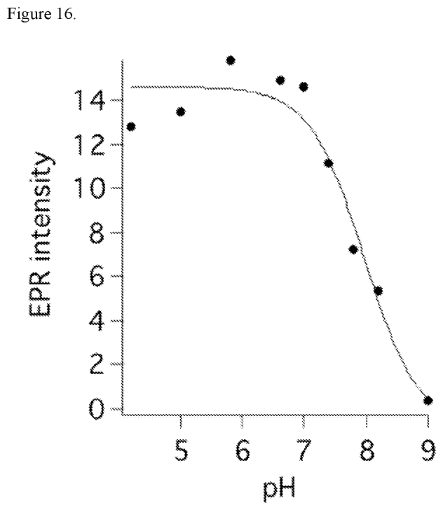

FIG. 15 shows the EPR signal at different pHs of 1 mM Fe-PyCy2AI (9.38 GHz, 10 K, 0.2 mW). The inset depicts the EPR signal between 65-90 mT with a contracted vertical scale. FIG. 16 shows the EPR signal intensity of 1 mM Fe-PyCy2AI at 150 mT at different pHs.

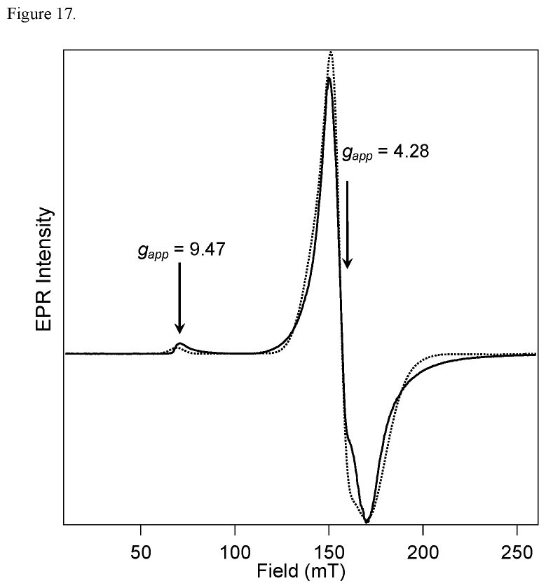

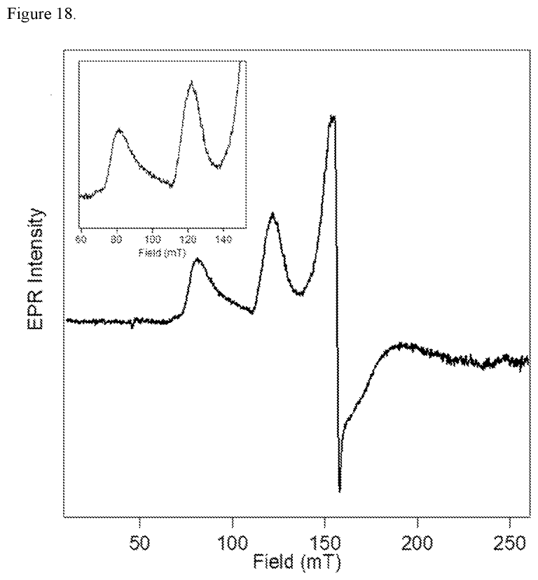

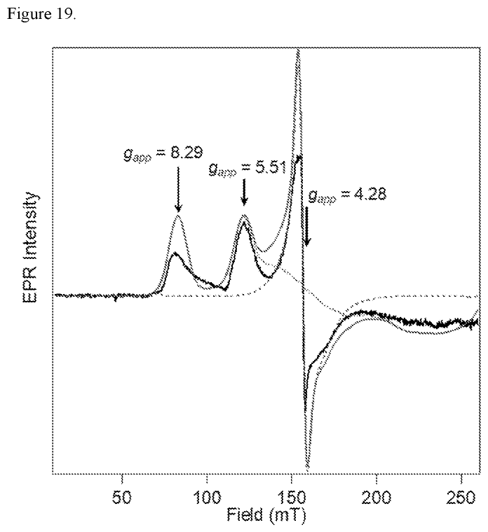

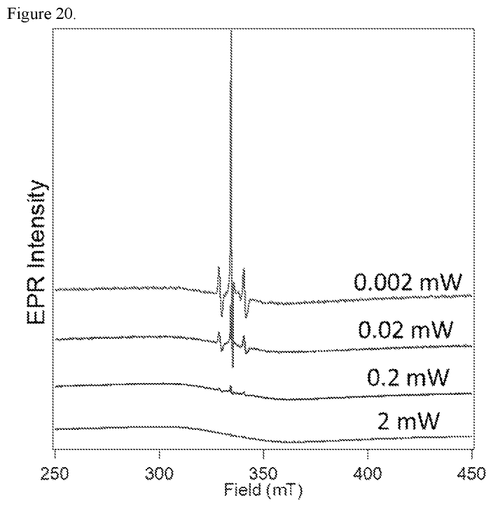

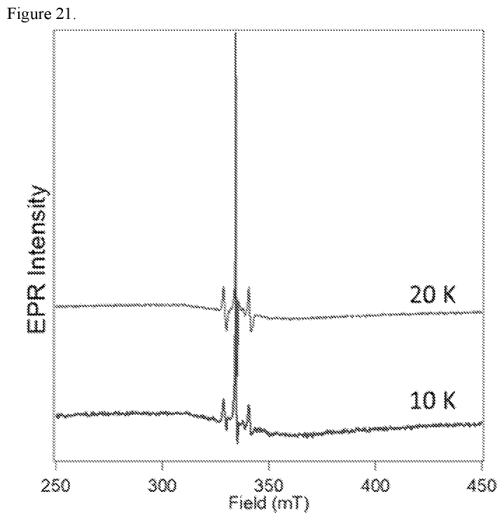

FIG. 17 shows a CW EPR spectrum recorded with 1 mM concentration of Fe-PyCy2AI at T=10 K and Pμw=0.2 mW at pH 4.2. The dotted line represents major component of simulation with E/D=0.28. g=1.95 g=2.06 g=1.99 D=3.7 cm-1 E=0. 94 cm-1 E/D=0.25. FIG. 18 shows CW X-band EPR spectrum recorded with 1 mM concentration of Fe-PyCy2AI at T=10 K and Pμw=0.2 mW at pH 9.0. The inset shows the low field region of the reported spectrum. FIG. 19 shows experimental data (black line) overlaid with the total simulation (gray line) for pH=9.0. The dotted line represents major component of simulation with an E/D=0.11, dashed line represents minor component of simulation with E/D=0.28. g=1.95 g=1.98 g=2.03 D=0.66 cm-1 E=0.19 cm-1 E/D=0.28 10% g=2.30 g=2.00 g=1.95 D=1.38 cm-1 E=0.15 cm-1 E/D=11 90%. FIG. 20 shows high field power dependence of Fe-PyCy2AI at pH=7.0. CW X-band EPR spectra were recorded at T=20 K and a concentration of 1 mM. The spectra are offset for clarity. FIG. 21 shows high field CW X-band EPR Spectra of 1 mM Fe-PyCy2AI at pH=7.0 at Pμw=0.002 mW and designated temperatures. The spectra are offset for clarity.

### X-ray Data Collection and Structure Solution Refinement

Single crystals of [Fe((PyCy2AI))₂O*6H₂O] ((ML)₂O) were obtained from 20-25 mM pH 6.7 and pH 9.3 solutions upon standing for 48 hours at room temperature. Dozens of orange square block crystals were obtained at pH 6.6, and two larger square blocks of orange crystals were obtained at pH 9.3. A 0.29×0.22×0.12 mM specimen from the pH 6.6 solution was selected for structural analysis. Unit cell analysis of the crystals isolated from the pH 9.3 sample were confirmed that the structures were identical.

Computing details: Data collection: APEX3 (Bruker, 2016); cell refinement: SAINT V8.40A; data reduction: SAINT V8.40A; program(s) used to solve structure: She1XT; program(s) used to refine structure: SHELXL; molecular graphics: Olex2; software used to prepare material for publication: Olex2.

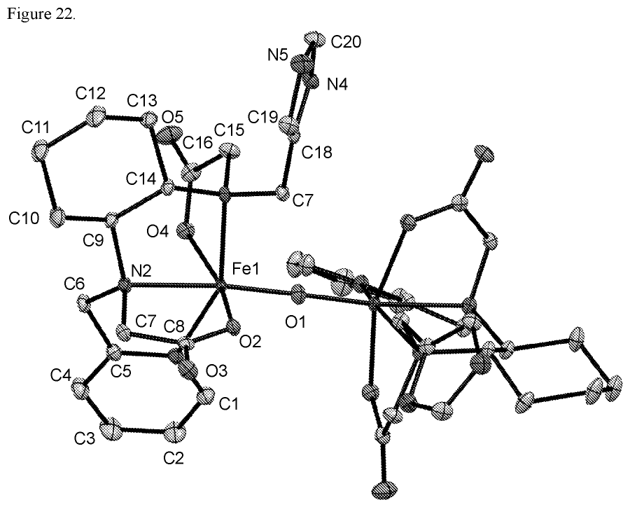

Crystal data, data collection parameters, and structure refinement details, and geometric parameters are shown in Tables 1A-1D. The (ML)₂O crystal structure with atoms labelled is shown in FIG. 22.

TABLE 1A
Crystal data for complex (ML)₂O.

| | |
|---|---|
| C₄₀H₅₀Fe₂N₁₀O₉•6(H₂O) | F(000) = 1208 |
| Mᵣ = 1142.79 | Dₓ = 1.465 Mg m⁻³ |
| Monoclinic, P2/c | Cu Kα radiation, λ = 1.54178 Å |
| a = 11.9411 (9) Å | Cell parameters from 9303 reflections |
| b = 9.7448 (7) Å | θ = 6.0-66.6° |
| c = 22.2881 (17) Å | μ = 5.23 mm⁻¹ |
| β = 92.422 (3)° | T = 100 K |
| V = 2591.2 (3) Å³ | Block, orange |
| Z = 2 | 0.29 × 0.22 × 0.12 mm |

TABLE 1B
X-ray data collection parameters for complex (ML)₂O.

| | |
|---|---|
| Bruker X8 Proteum-R diffractometer | 4586 independent reflections |
| Radiation source: rotating anode | 4562 reflections with I > 2σ(I) |
| Montel monochromator | Rᵢₙₜ = 0.043 |
| ϕ and ω scans | θₘₐₓ = 66.7°, θₘᵢₙ = 5.0° |
| Absorption correction: multi-scan | h = −14→14 |
| SADABS2016/2 (Bruker, 2016/2) was | |
| used for absorption correction. wR2(int) | |
| was 0.1079 before and 0.0619 after | |
| correction. The Ratio of minimum to | |
| maximum transmission is 0.7885. The | |
| □/2 correction factor is Not present. | |
| Tₘᵢₙ = 0.594, Tₘₐₓ = 0.753 | k = −11→11 |
| 90513 measured reflections | l = −26→26 |

TABLE 1C
Crystal structure refinement of complex (ML)₂O.

| | |
|---|---|
| Refinement on F² | Primary atom site location: dual |
| Least-squares matrix: full | Hydrogen site location: mixed |
| R[F² > 2σ(F²)] = 0.031 | H atoms treated by a mixture of |
| | independent and constrained refinement |
| wR(F²) = 0.082 | w = 1/[σ²(Fₒ²) + (0.0447P)² + 1.8917P] |
| | where P = (Fₒ² + 2F_{c}²)/3 |
| S = 1.08 | (Δ/σ)ₘₐₓ = 0.002 |
| 4586 reflections | Δ>ₘₐₓ = 0.78 e Å⁻³ |
| 359 parameters | Δ>ₘᵢₙ = −0.34 e Å⁻³ |
| 0 restraints | |

Special details
Geometry. All esds (except the esd in the dihedral angle between two l.s. planes) are estimated using the full covariance matrix. The cell esds are taken into account individually in the estimation of esds in distances, angles and torsion angles; correlations between esds in cell parameters are only used when they are defined by crystal symmetry. An approximate (isotropic) treatment of cell esds is used for estimating esds involving l.s. planes.

TABLE 1D
Geometric parameters for (ML)₂O crystal structure.

| | | | | | |
|---|---|---|---|---|---|
| Fe1—O4 | 2.0335 | (12) | N2—C7 | 1.476 | (2) |
| Fe1—O2 | 2.0332 | (12) | N5—C19 | 1.371 | (2) |
| Fe1—O1 | 1.7791 | (3) | N5—C20 | 1.341 | (2) |
| Fe1—N3 | 2.2089 | (13) | C14—C9 | 1.542 | (2) |
| Fe1—N1 | 2.1295 | (14) | C14—C13 | 1.541 | (2) |
| Fe1—N2 | 2.2229 | (14) | C17—C18 | 1.493 | (2) |
| O4—C16 | 1.278 | (2) | C19—C18 | 1.367 | (2) |
| O2—C8 | 1.275 | (2) | C9—C10 | 1.536 | (2) |
| O3—C8 | 1.242 | (2) | C16—C15 | 1.524 | (2) |
| O5—C16 | 1.231 | (2) | C8—C7 | 1.516 | (2) |
| N3—C14 | 1.5084 | (19) | C5—C6 | 1.507 | (2) |
| N3—C17 | 1.506 | (2) | C5—C4 | 1.384 | (2) |
| N3—C15 | 1.488 | (2) | C1—C2 | 1.377 | (3) |
| N1—C5 | 1.346 | (2) | C10—C11 | 1.526 | (2) |
| N1—C1 | 1.346 | (2) | C13—C12 | 1.528 | (2) |
| N4—C18 | 1.390 | (2) | C12—C11 | 1.517 | (3) |
| N4—C20 | 1.324 | (2) | C4—C3 | 1.387 | (3) |
| N2—C9 | 1.505 | (2) | C3—C2 | 1.381 | (3) |
| N2—C6 | 1.485 | (2) | | | |
| O4—Fe1—N3 | 79.46 | (5) | C20—N5—C19 | 107.16 | (14) |
| O4—Fe1—N1 | 82.52 | (5) | N3—C14—C9 | 112.79 | (12) |
| O4—Fe1—N2 | 84.06 | (5) | N3—C14—C13 | 114.70 | (13) |
| O2—Fe1—O4 | 161.06 | (5) | C13—C14—C9 | 107.02 | (13) |
| O2—Fe1—N3 | 89.59 | (5) | C18—C17—N3 | 116.11 | (13) |
| O2—Fe1—N1 | 101.80 | (5) | C18—C19—N5 | 106.44 | (15) |
| O2—Fe1—N2 | 79.20 | (5) | N4—C18—C17 | 122.14 | (14) |
| O1—Fe1—O4 | 101.02 | (5) | C19—C18—N4 | 109.21 | (14) |
| O1—Fe1—O2 | 96.25 | (5) | C19—C18—C17 | 128.65 | (15) |
| O1—Fe1—N3 | 101.43 | (5) | N2—C9—C14 | 113.04 | (13) |
| O1—Fe1—N1 | 101.19 | (6) | N2—C9—C10 | 112.98 | (14) |
| O1—Fe1—N2 | 173.98 | (4) | C10—C9—C14 | 109.69 | (13) |
| N3—Fe1—N2 | 82.59 | (5) | O4—C16—C15 | 115.74 | (14) |
| N1—Fe1—N3 | 153.33 | (5) | O5—C16—O4 | 123.19 | (16) |
| N1—Fe1—N2 | 76.11 | (5) | O5—C16—C15 | 121.04 | (15) |
| C16—O4—Fe1 | 118.12 | (10) | O2—C8—C7 | 116.89 | (14) |
| C8—O2—Fe1 | 118.98 | (10) | O3—C8—O2 | 124.51 | (15) |
| Fe1ⁱ—O1—Fe1 | 177.38 | (10) | O3—C8—C7 | 118.51 | (14) |
| C14—N3—Fe1 | 108.51 | (9) | N1—C5—C6 | 116.90 | (14) |
| C17—N3—Fe1 | 107.85 | (9) | N1—C5—C4 | 121.24 | (16) |
| C17—N3—C14 | 111.47 | (12) | C4—C5—C6 | 121.73 | (15) |
| C15—N3—Fe1 | 103.23 | (9) | N1—C1—C2 | 121.91 | (16) |
| C15—N3—C14 | 114.44 | (13) | N2—C6—C5 | 110.39 | (13) |
| C15—N3—C17 | 110.80 | (12) | C11—C10—C9 | 111.98 | (15) |
| C5—N1—Fe1 | 115.94 | (11) | N2—C7—C8 | 112.83 | (13) |
| C1—N1—Fe1 | 123.49 | (11) | C12—C13—C14 | 110.70 | (14) |
| C1—N1—C5 | 119.45 | (14) | N3—C15—C16 | 111.39 | (13) |
| C20—N4—C18 | 105.00 | (14) | N4—C20—N5 | 112.18 | (15) |
| C9—N2—Fe1 | 107.58 | (9) | C11—C12—C13 | 110.51 | (15) |
| C6—N2—Fe1 | 107.42 | (9) | C5—C4—C3 | 119.17 | (17) |
| C6—N2—C9 | 111.10 | (13) | C12—C11—C10 | 110.03 | (15) |
| C7—N2—Fe1 | 105.47 | (9) | C2—C3—C4 | 119.22 | (17) |
| C7—N2—C9 | 115.71 | (12) | C1—C2—C3 | 118.98 | (17) |
| C7—N2—C6 | 109.10 | (13) | | | |

### Example 4. Estimation of log K from EDTA competition assay

The [Fe(PyCy2AI)]⁺ formation constant was estimated from direct competition with EDTA at pH 4.4, I=0.1 M KNO₃, and 310 K. Product distribution at equilibrium was estimated from an HPLC analysis of the reaction mixture (HPLC method A1). The equilibrium concentrations of [Fe(PyCy2AI)]⁺ were quantified according to a linear calibration plot of HPLC peak area as a function of chelate concentration. An equilibrium constant, K_{eq}, was estimated using Eq 4A,

$${K}_{e q} = \frac{[ PyCy 2 AI ] [ Fe - EDTA ]}{[ Fe - PyCy 2 AI ] [ E D T A ]} . \qquad ( Eq 4 A )$$

The conditional (pH 4.4) formation constant, K_{cond}, was estimated from K_{eq} and the previously (K_{cond} of Fe-EDTA measured under identical conditions using Eq 4B,

$${K}_{e q} = \frac{{K}_{cond Fe - E D T A}}{{K}_{cond Fe - PyCy 2 AI}} . \qquad ( Eq 4 B )$$

Under HPLC experimental conditions that cannot distinguish between chemical forms of the complex, the conditional stability constant can be estimated from the concentration coefficient of the ligand “L”, or PyCy2AI²⁻, species (α_{L}), which is known from the ligand pH-titration data. The formation constant was thus estimated using Eq 4C,

K_{cond}=α_{L}K (Eq 4C).

The HPLC assay conditions were chosen specifically to minimize contributions from ‘side products’ such as [Fe(HPyCy2AI)]²⁺, [Fe(PyC2AI)(OH)], or [Fe(HEDTA)], which together we estimate to contribute <5% and <2% of total PyCy2AI and EDTA speciation.

### Example 5. Bulk magnetic susceptibility

The effective magnetic moment (1 μ_{eff}) was estimated using the modified Evans method based on ¹H NMR at 310 K. Fe complex was dissolved in H₂O doped with 5% tert-butanol by volume and placed in a WILMAD coaxial insert tube containing blank solvent doped with 5% tert-butanol as diamagnetic reference. The molar susceptibilities were determined from the difference in tert-butanol chemical shift between the Fe complex solution and diamagnetic reference according to Eq 5A and Eq 5B,

$${χ}_{M} = {χ}_{g} \times M W \qquad ( Eq 5 A )$$

$${χ}_{g} = \vert \frac{- 3 Δ f}{4 π fm} + {χ}_{0} \vert \qquad ( Eq 5 B )$$

where X_{g} and Xₘ are the mass susceptibility and molar susceptibility of the Fe complex, m is the concentration of the Fe complex in g/mL, Δƒ is the separation between the resonance frequencies of tert-butanol between the Fe complex solution and diamagnetic reference (in Hz), and ƒ is Larmor frequency of water ¹H at the field strength of the spectrometer (499.8 MHz), and X₀ is the mass susceptibility of pure solvent (X₀=−0.7203×10⁻⁶ mL/g for H₂O). The molar susceptibility relates directly to Leff by Eq 5C, where T is temperature,

μ_{eff}=2.84\sqrt{XM^{T}} (Eq 5C).

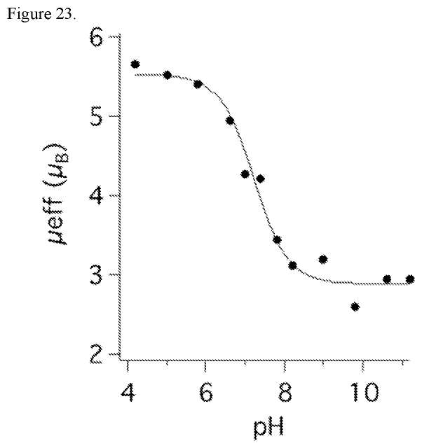

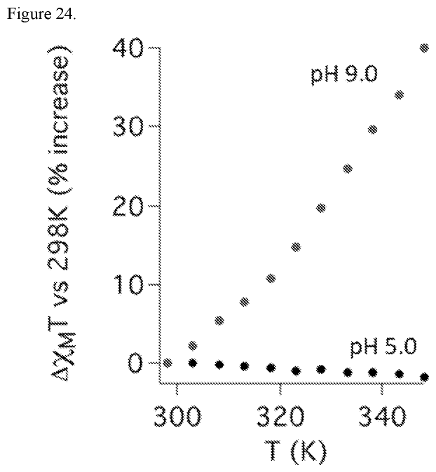

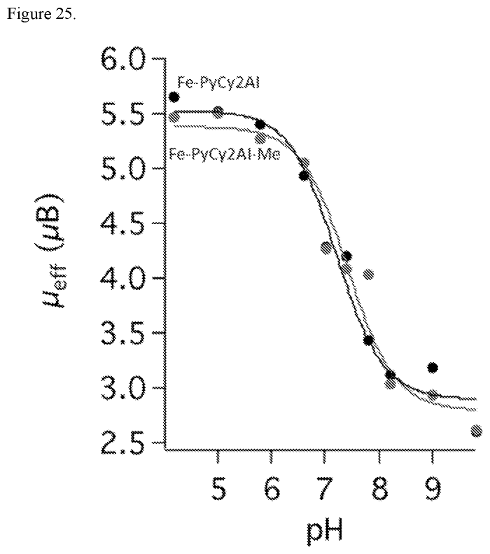

FIG. 23 shows bulk magnetic susceptibility at different pHs of 10 mM Fe-PyCy2AI at 310 K. FIG. 24 shows bulk magnetic susceptibility at different temperatures of 10 mM Fe-PyCy2AI at pH 5.0 and pH 9.0. FIG. 25 shows bulk magnetic susceptibility at different pHs of mM Fe-PyCy2AI and 10 mM Fe-PyCy2AI-Me at 310 K.

### Example 6. Relaxivity Measurements

Relaxivity measurements were performed on a Bruker mq60 Minispec, 1.41 T, and on an 11.7 T Varian NMR spectrometer at 37° C. Longitudinal (T₁) relaxation times were measured via an inversion recovery experiment using 10 inversion times of duration ranging between 0.05 s T₁ and 5 s T₁. Transverse (T₂) relaxation times were measured using a Carl-Purcell-Meiboom-Gill spin-echo experiment. Under experimental conditions where 1/T_{1,2} increased linearly with increasing [Fe], relaxivity (r₂i=1,2) was determined from the slope of a plot of 1/Tᵢ(i=1,2) vs. [Fe] for at least 4 concentrations. Under conditions where non-negligible dimerization occurs, plots of 1/T_{1,2} vs. [Fe] are non-linear and r_{1,2} were estimated by dividing the Fe induced increase in 1/T_{1,2} by [Fe]. The transverse (T₂) relaxation times of ¹⁷O were acquired at 11.7 T using a CPMG pulse sequence at temperatures ranging from 298 to 358K. Samples were prepared in neat H₂O adjusted and enriched with ˜0.5% v/v of H₂¹⁷O. The data were fit with the Swift-Connick expressions describing two-site exchange, as described previously.

The proposed pH-dependence on Fe-PyCy2AI speciation governing ri between pH 6.0 and pH 7.4 is shown below:

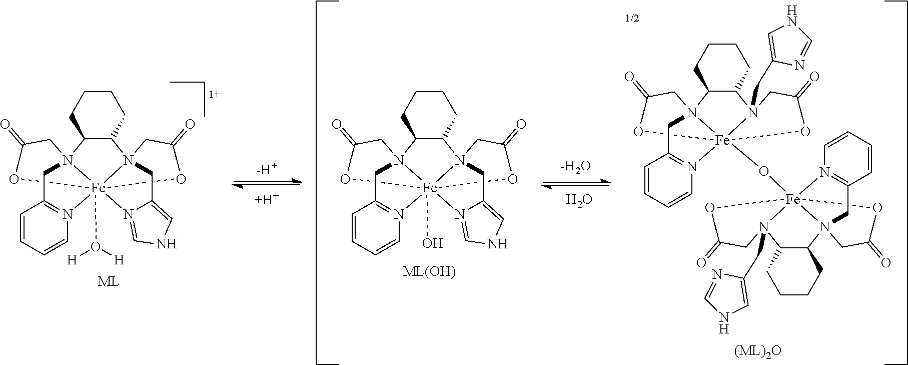

Table 6A shows the r₁ of ML, ML(OH), (ML)₂O at 310 K.

TABLE 6A

| | r₁ (mM⁻¹s⁻¹)/Fe | | |
|---|---|---|---|
| | 1.4 T | 4.7 T | 11.7 T |
| ML | 1.4 ± 0.01 | 1.9 ± 0.02 | 1.4 ± 0.05 |
| ML(OH) | 0.90 ± 0.03 | 1.7 ± 0.22 | 1.4 ± 0.25 |
| (ML)₂O | 0.072 ± 0.024 | 0.14 ± 0.058 | 0.20 ± 0.042 |

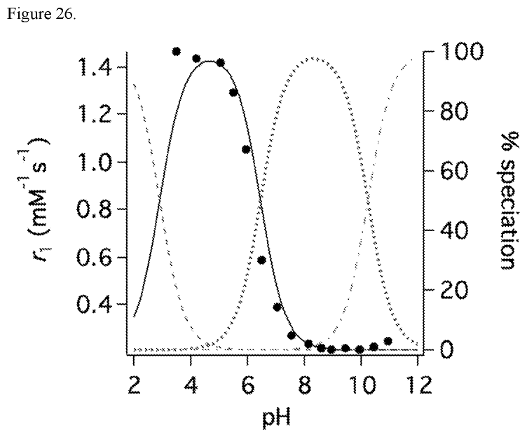

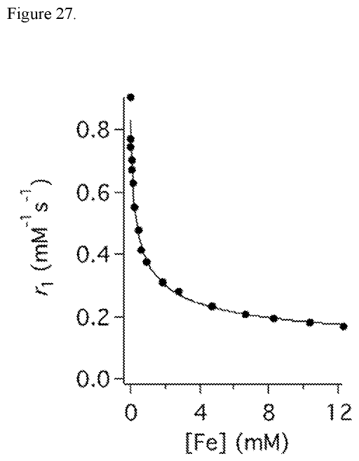

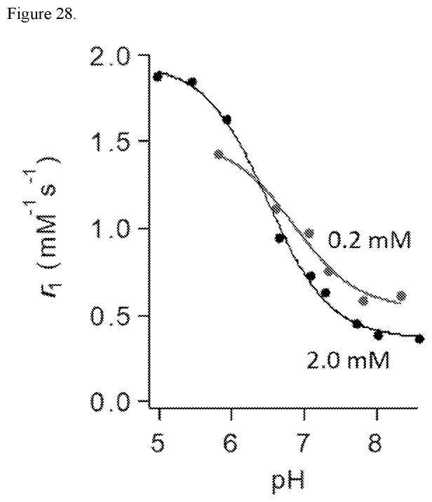

FIG. 26 shows overlay of Fe-PyCy2AI r₁ and the Fe-PyCy2AI protonation state as a function of pH. The red trace corresponds to percentage speciation of monoprotonated complex (HML), the black trace corresponds to the complex ML, the blue trace corresponds to the mono-deprotonated complex comprising a mixture of interconvertible species ML(OH) and (ML)₂O, and the green trace corresponds to the doubly deprotonated complex of tentative empirical formula MLH₋₂. At pH 2.0, free Fe comprises <0.02% of total Fe speciation. FIG. 27 shows r₁ of Fe-PyCy2AI at 310 K and 1.4 T as a function of Fe concentration. FIG. 28 shows r₁ of 0.2 mM and 2.0 mM solutions of Fe-PyCy2AI in human blood plasma at 1.4 T and 310 K as a function of pH.

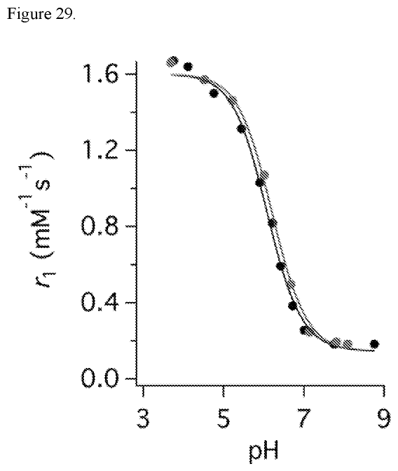

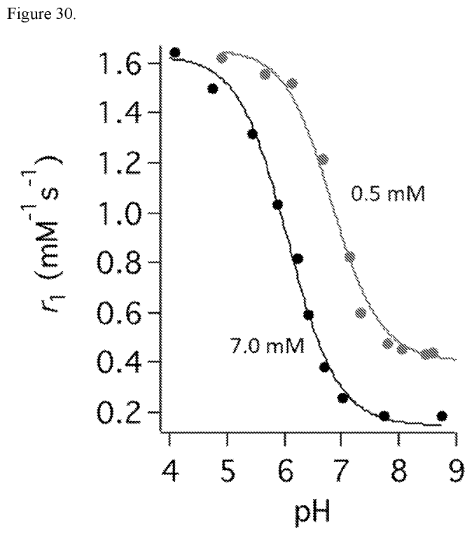

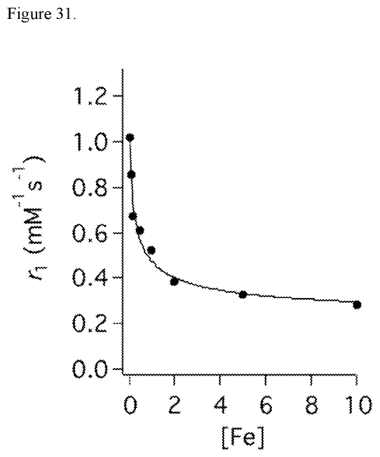

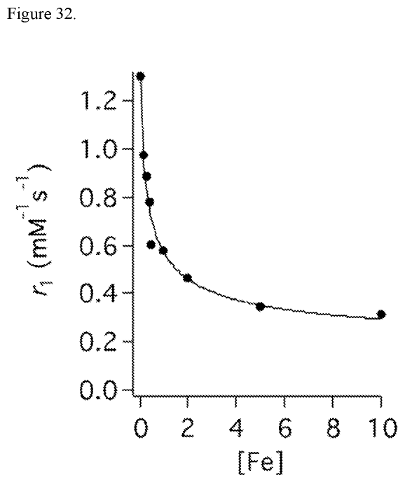

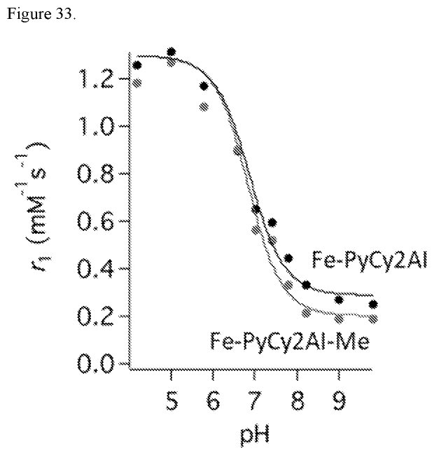

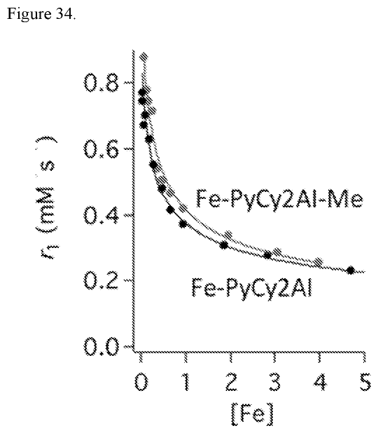

FIG. 29 shows plots of r₁ vs. pH of 7.0 mM Fe-PyCy2AI as pH is increased from pH 3.8 to pH 8.8 and then titrated in the reverse direction. This demonstrates that pH-dependence on r₁ is governed by an equilibrium mixture of Fe species. FIG. 30 shows r₁ of 7.0 mM and 0.5 mM solutions of Fe-PyCy2AI as functions of pH. The plots demonstrate the concentration dependence on apparent pKa of water co-ligand. FIG. 31 show the ri of Fe-PyCy2AI at pH 9.0, 310 K, and 4.7 T as a function of Fe concentration. FIG. 32 shows the ri of Fe-PyCy2AI at pH 9.0, 310 K, and 11.7 T as a function of Fe concentration. FIG. 33 shows r₁ of 10 mM Fe-PyCy2AI and 10 mM Fe-PyCy2AI-Me at 310 K and 1.4 T as a function of pH. FIG. 34 shows r₁ of Fe-PyCy2AI and Fe-PyCy2AI-Me at 310 K and 1.4 T as a function of Fe concentration.

### Example 7. Dissociation constant

The dissociation constant for (ML)₂O formation was estimated from the concentration dependence on r₁ at pH 9.0, under these experimental conditions, complexes speciation is comprised near entirely by ML(OH) and the corresponding (ML)₂O dimer and r₁ can be expressed as Eq 7A,

r₁=r₁^{mon}αₘₒₙ+r₁^{dim}α_{dim} (Eq 7A)

where r₁^{mon} and r₁^{dim} are the r₁ corresponding to ML(OH) and (ML)₂O and αₘₒₙ and α_{dim} are the fraction of total [Fe] comprised by each.

The concentration dependence on monomer vs. dimer speciation is governed by the equilibrium constant (K_{d}) in Eq 7B,

$${K}_{d} = \frac{[ mon ] [ mon ]}{[ dimer ]} = \frac{( [ Fe ] - 2 [ dimer ] ) ( [ Fe ] - 2 [ dimer ] )}{[ dimer ]} . \qquad ( Eq 7 B )$$

Eq 7B can be rearranged to the corresponding quadratic equation, Eq 7C, and concentration of dimer solved by Eq 7D,

$$0 = {4 [ Fe ]}^{2} - [ dim ] ( 4 [ Fe ] + {K}_{d} ) + {4 [ dim ]}^{2} \qquad ( Eq 7 C )$$

$$[ dim ] = \frac{( 4 [ Fe ] + {K}_{d} ) - \sqrt{{( 4 [ Fe ] + {K}_{d} )}^{2} - {16 [ Fe ]}^{2}}}{8} . \qquad ( Eq 7 D )$$

Thus, the concentration dependence on ri was fit to a 3 parameter fit based on Eqs 7A-7D, yielding estimates of r₁^{mon}, r₁^{dim}, and K_{d}.

### Example 8. Magnetic Resonance Imaging

Magnetic resonance imaging was performed using a Bruker Biospec 4.7 T system. Samples were placed in a homemade sample holder and imaged using a volume coil. The sample holder accommodates simultaneous measurement of up to 60 samples. The sample was incubated at 310 K by continuously blowing warm air into the scanner bore. Temperature inside the scanner was confirmed using a non-magnetic MR compatible thermometer. T₁ values for each compound was determined using a 2D rapid acquisition refocused echo (RARE) sequence of flip angle (FA)=90°, echo time (TE)=20 ms, repetition time (TR) ranging from 146-7500 ms, acquisition matrix=256×128, field of view=64×32 mm, slice thickness 1 mm. T₁ measurements were performed on samples ranging between 0.05-2 mM Fe³⁺-PyCy2AI. T₁ was obtained from a nonlinear least square fit of the signal intensity (SI(t)) vs TR curve where T₁ and α are adjustable parameters, Eq 8A,

$$SI ( t ) = \frac{a ( 1 - {e}^{- T R / T 1} )}{1 - {e}^{- T R / T 1} cos ( FA )} = a ( 1 - {e}^{- T R / T 1} ) . \qquad ( Eq 8 A )$$

T₁-weighted images were obtained using a 2D RARE spin echo sequence: TR/TE/FA=250 ms/6.33 ms/ 90°; acquisition matrix=256×256, field of view=45×45 mm, slice thickness 1 mm.

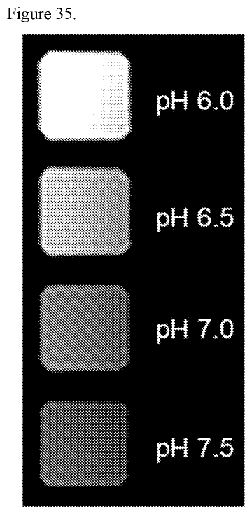

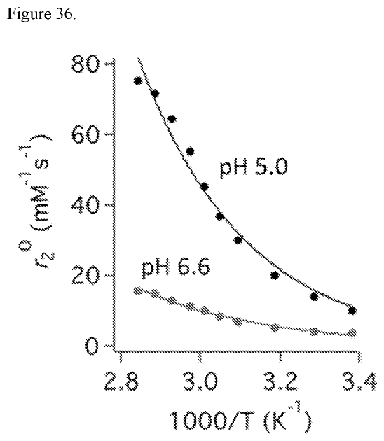

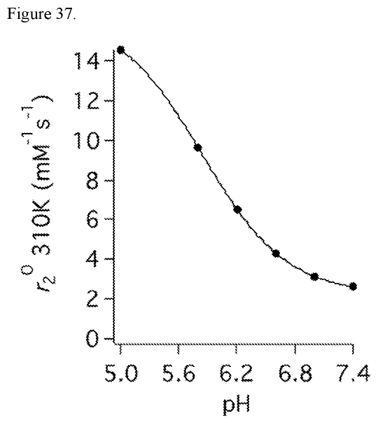

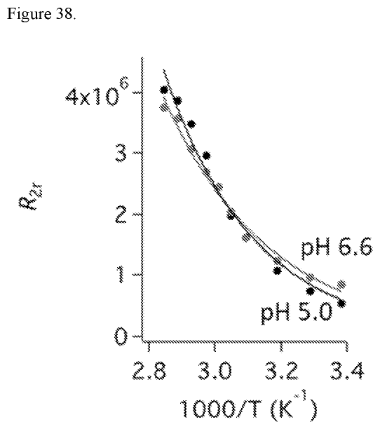

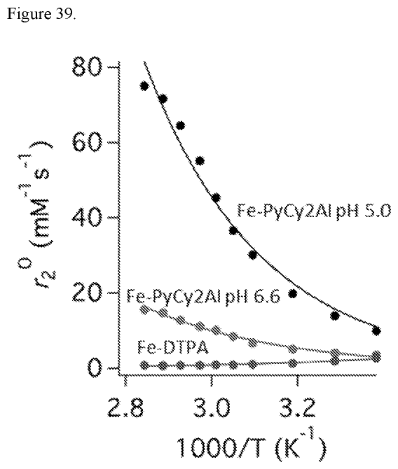

FIG. 35 shows T₁-weighted 2D spin echo images (TR=250 ms, TE=6.33 ms, FA=90°) of phantoms containing 2.0 mM Fe-PyCy2AI adjusted to different pH values between pH 6.0-7.5, 4.7 T, and 310 K. FIG. 36 shows T₂-relaxivity of water ¹⁷O (r₂^{O}) in the presence of 75 mM Fe-PyCy2AI at pH 5.0 or pH 6.6 as a function of temperature. FIG. 37 shows r₂^{O} at 310 K of Fe-PyCy2AI as a function of pH. FIG. 38 shows ¹⁷O T₂-relaxation rate as a function of temperature normalized to the mole fraction of water molecules coordinated to Fe³⁺ (R₂ᵣ) assuming q=1 at pH 5.0 and q=0.23 at pH 6.6, which are estimated from equilibrium data in Tables 8A and 8B. FIG. 39 shows T₂-relaxivity of water ¹⁷O (r₂^{O}) in the presence of Fe-PyCy2AI at pH 5.0, Fe-PyCy2AI at pH 6.6, and Fe-DTPA at pH 7.4.

Table 8A shows protonation and formation constants of ligands and corresponding Fe³⁺ complexes.

TABLE 8A

| | log | log | log | log | log | log | log | log |
|---|---|---|---|---|---|---|---|---|
| Ligand (L) | K_{LH} | K_{LH2} | K_{LH3} | K_{LH4} | K_{ML} | K_{HML} | K_{ML/ML(OH)}^{e,f} | K_{MLH-1/MLH-2} |
| PyCy2AI^{a,b} | 10.62 ± 0.04 | 6.44 ± 0.03 | 4.23 ± 0.033 | 2.27 ± 0.05 | 23.9 ± 0.03^{c} | 2.91 ± 0.05 | 6.46 ± 0.03 | 10.24 ± 0.05 |
| EDTA^{d} | 10.19 | 6.13 | 2.69 | 2.00 | 25.1 | 1.3 | 7.37 | — |
| CyDTA^{d} | 12.3 | 6.12 | 3.49 | 2.40 | 29.1 | — | 9.57 | — |
| PyC3A^{d} | 10.16 | 6.39 | 3.13 | — | 26.0^{c} | — | 8.50^{g} | — |

ªIsolated in H₄PyCy2AI form where H corresponds to ionizable protons.
^{b}Values obtained by pH-potentiometry ligand (0.1 M NaCl, 310 K). K_{LHn} defined as [HₙL]/[H][Hₙ₋₁L].
^{c}Value estimated from K_{comp} using EDTA challenge. K_{ML} defined as [ML]/[M][L].
^{d}Values obtained by pH-potentiometry of 7.0 mM solution of ligand and Fe³⁺ (0.1 M NaCl, 310 K). K_{HML} defined as [HML]/[H][ML]; K_{ML/ML(OH)} defined as [ML]/[ML(OH)][H].
^{e}ML(OH) of PyCy2AI, EDTA, and CyDTA are known to exist in equilibrium with (ML)₂O species as shown in example 6. The equilibrium mixture can also be referred to as MLH₋₁. K_{MLH-1/MLH-2} defined as [MLH-1]/[MLH-₂][H].
^{f}K_{ML/ML(OH)} is not an absolute protonation constant and corresponds specifically to that observed under single set of titration conditions of 7.0 mM each Fe and ligand. hMLH₋₂ is the tentatively assigned empirical formula of the doubly deprotonated complex.
^{g}Estimated from pH dependence on r₁.

Table 8B shows Ka for dimerization for Fe³⁺ complexes.

TABLE 8B

| | Dimerization Kₐ (mM⁻¹) |
|---|---|
| PyCy2AI | 2.3 ± 0.57,^{a} 5.1 ± 2.7,^{b} 7.4 ± 5.5,^{c} 6.2 ± 1.5^{d} |
| EDTA | 0.44 |
| PDTA^{e} | 0.19 |
| CyDTA | 0.012 |
| HEDTA | 0.24 |

^{a-c}Determined from concentration dependence on relaxivity at pH 9.0 and 310 K at 1.4 T, 4.7 T, and 11.7 T, respectively.
^{d}Determined from concentration dependence on extinction coefficient at 342 nm at pH 9.0 and 310 K.
^{e}PDTA = N,N,N′,N′-1,2-propylenediaminetetracetic acid.

What is claimed is:

1. A compound of Formula (I) 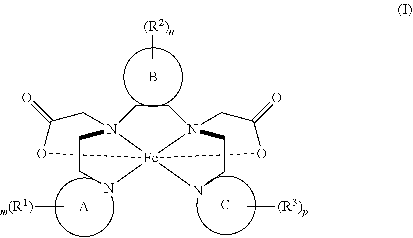
or a pharmaceutically acceptable salt thereof, wherein:
Ring A is a 5-10 membered heteroaryl or a 5-10 membered heterocycloalkyl;
Ring B is phenyl or a C₅₋₇ cycloalkyl;
Ring C is a 5-10 membered heteroaryl or a 5-10 membered heterocycloalkyl;
each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};
m, n, and p are each independently 0, 1, 2, or 3;
each q is independently 0 or 1;
each R^{A} and R^{B} are independently hydrogen or C₁₋₆ alkyl; and
each R^{C} is independently C₁₋₆ alkyl.

2. A compound of Formula (II) 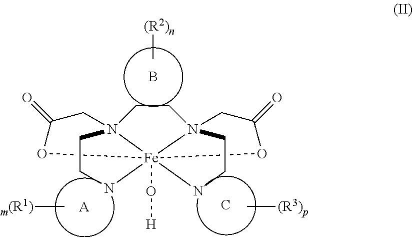
or a pharmaceutically acceptable salt thereof, wherein:
Ring A is a 5-10 membered heteroaryl or a 5-10 membered heterocycloalkyl;
Ring B is phenyl or a C5-7 cycloalkyl;
Ring C is a 5-10 membered heteroaryl or a 5-10 membered heterocycloalkyl;
each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};
m, n, and p are each independently 0, 1, 2, or 3;
each q is independently 0 or 1;
each R^{A} and R^{B} are independently hydrogen or C₁₋₆ alkyl; and
each R^{C} is independently C₁₋₆ alkyl.

3. A compound of Formula (III) 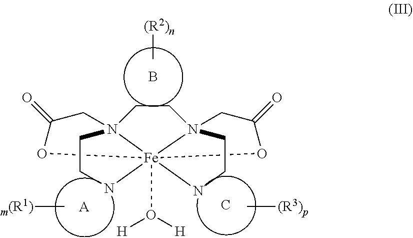
or a pharmaceutically acceptable salt thereof, wherein:
Ring A is a 5-10 membered heteroaryl or a 5-10 membered heterocycloalkyl;
Ring B is phenyl or a C₅₋₇ cycloalkyl;
Ring C is a 5-10 membered heteroaryl or a 5-10 membered heterocycloalkyl;
each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O )R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};
m, n, and p are each independently 0, 1, 2, or 3;
each q is independently 0 or 1;
each R^{A} and R^{B} are independently hydrogen or C₁₋₆ alkyl; and
each R^{C} is independently C₁₋₆ alkyl.

4. A compound of Formula (IV) 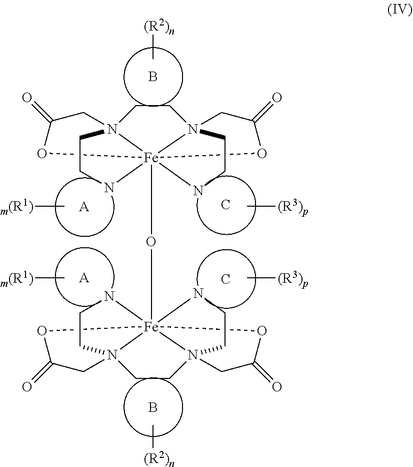
or a pharmaceutically acceptable salt thereof, wherein:
Ring A is a 5-10 membered heteroaryl or a 5-10 membered heterocycloalkyl;
Ring B is phenyl or a C₅₋₇ cycloalkyl;
Ring C is a 5-10 membered heteroaryl or a 5-10 membered heterocycloalkyl;
each R¹, R², and R³ is independently halogen, hydroxyl, cyano, C₁₋₆ alkyl, C₂₋₆ alkenyl, C₁₋₆ alkynyl, C₁₋₆ hydroxyalkyl, C₁₋₆ haloalkyl, C₁₋₆ alkoxy, C₁₋₆ haloalkoxy, —(C₁₋₆ alkyl)_{q}SO₃H, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}NR^{A}(C═O)R^{C}, 4-6 membered heterocycloalkyl, 5-6 membered heteroaryl, C₃₋₆ cycloalkyl, —(C₁₋₆ alkyl)_{q}SO₂R^{A}, —(C₁₋₆ alkyl)_{q}NHSO₂R^{A}, —(C═O)NHSO₂R^{A}, —P(R^{A})O₂R^{B}, and —PO₃R^{A}R^{B};
m, n, and p are each independently 0, 1, 2, or 3;
each q is independently 0 or 1;
each R^{A} and R^{B} are independently hydrogen or C₁₋₆ alkyl; and
each R^{C} is independently C₁₋₆ alkyl.

5. The compound of any one of claims 1-4, wherein Ring A is a 5-10 membered heterocycloalkyl.

6. The compound of any one of claims 1-4, wherein Ring A is a 5-10 membered heteroaryl.

7. The compound of any one of claims 1-4, wherein Ring A is a 5-6 membered heteroaryl.

8. The compound of any one of claims 1-4, wherein Ring A is selected from

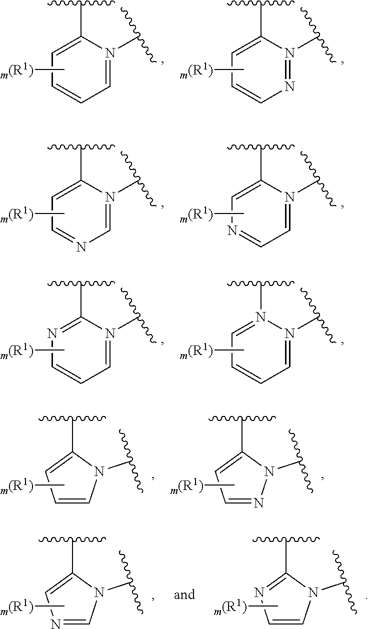

9. The compound of any one of claims 1-8, wherein m is 0.

10. The compound of any one of claims 1-8, wherein m is 1, 2, or 3.

11. The compound of any claim 10, wherein R¹ is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

12. The compound of any one of claims 1-11, wherein Ring B is phenyl.

13. The compound of any one of claims 1-11, wherein Ring B is C₅₋₇ cycloalkyl.

14. The compound of any one of claims 1-11, wherein Ring B is C₆ cycloalkyl.

15. The compound of any one of claims 1-14, wherein n is 0.

16. The compound of any one of claims 1-14, wherein n is 1, 2, or 3.

17. The compound of claim 16, wherein R² is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

18. The compound of any one of claims 1-17, wherein Ring C is 5-10 membered heterocycloalkyl.

19. The compound of any one of claims 1-17, wherein Ring C is 5-6 membered heterocycloalkyl.

20. The compound of any one of claims 1-17, wherein Ring C is selected from

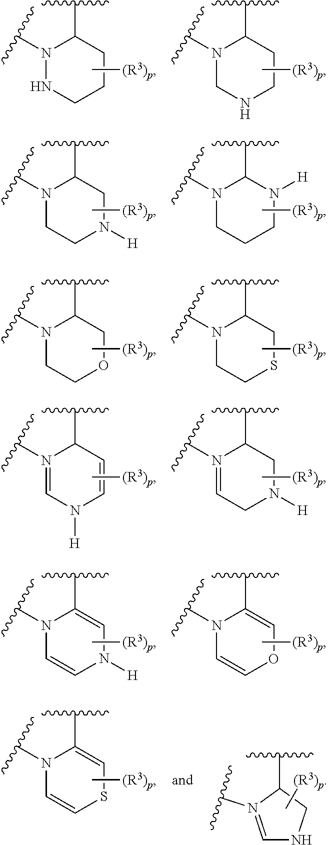

21. The compound of any one of claims 1-17, wherein Ring C is 5-10 membered heteroaryl.

22. The compound of any one of claims 1-17, wherein Ring C is 5-6 membered heteroaryl.

23. The compound of any one of claims 1-17, wherein Ring C is selected from

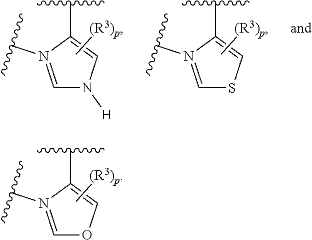

24. The compound of any one of claims 1-23, wherein p is 0.

25. The compound of any one of claims 1-23, wherein p is 1, 2, or 3.

26. The compound of claim 25, wherein R³ is halogen, hydroxyl, cyano, C₁₋₆ alkyl C₁₋₆ hydroxyalkyl, C₁₋₆ haloalkyl, C₁₋₆ alkoxy, or C₁₋₆ haloalkoxy.

27. The compound of claim 1, wherein the compound is 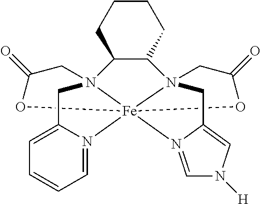
or a pharmaceutically acceptable salt thereof.

28. The compound of claim 2, wherein the compound is 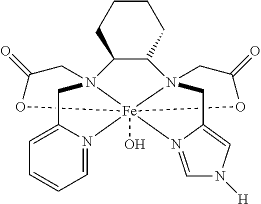
or a pharmaceutically acceptable salt thereof.

29. The compound of claim 3, wherein the compound is 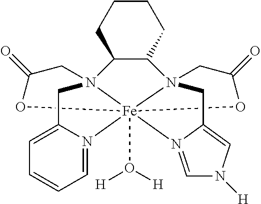
or a pharmaceutically acceptable salt thereof.

30. The compound of claim 4, wherein the compound is 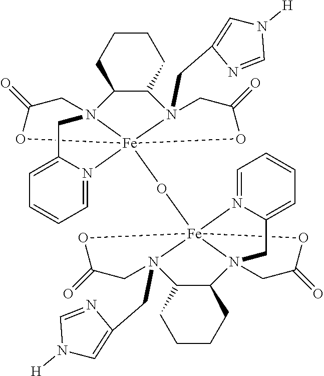
or a pharmaceutically acceptable salt thereof.

31. The compound of any one of claim 1, 3, or 5-30, wherein the relaxivity of the compound is about 1.0 to about 5.0.

32. The compound of any one of claim 1, 3, or 5-31, wherein the relaxivity of the compound is about 1.0 to about 3.0.

33. The compound of any one of claim 1, 3, or 5-32, wherein the relaxivity of the compound is about 1.0 to about 2.5.

34. The compound of any one of claim 1, 3, or 5-33, wherein the relaxivity of the compound is about 1.2 to about 2.3.

35. The compound of any one of claim 1, 3, or 5-34, wherein the relaxivity of the compound is about 1.3 to about 2.0.

36. The compound of any one of claim 1, 3, or 5-35, wherein the relaxivity of the compound is about 1.4 or about 1.9.

37. The compound of any one of claim 2 or 5-30, wherein the relaxivity of the compound is about 0.6 to about 4.5.

38. The compound of any one of claim 2, 5-30, or 37, wherein the relaxivity of the compound is about 0.6 to about 4.0.

39. The compound of any one of claim 2, 5-30, or 37-38, wherein the relaxivity of the compound is about 0.6 to about 2.0.

40. The compound of any one claim 2, 5-30, or 37-39, wherein the relaxivity of the compound is about 0.7 to about 1.9.

41. The compound of any one of claim 2, 5-30, or 37-40, wherein the relaxivity of the compound is about 0.8 to about 1.8.

42. The compound of any one of claim 2, 5-30, or 37-41, wherein the relaxivity of the compound is about 0.9, about 1.4 or about 1.7.

43. The compound of any one of claim 3 or 5-30, wherein the relaxivity of the compound is about 0.02 to about 0.50.

44. The compound of any one of claim 4-30 or 43, wherein the relaxivity of the compound is about 0.03 to about 0.45.

45. The compound of any one of claim 4-30 or 43-44, wherein the relaxivity of the compound is about 0.05 to about 0.30.

46. The compound of any one of claim 4-30 or 43-45, wherein the relaxivity of the compound is about 0.06 to about 0.30.

47. The compound of any one claim 4-30 or 43-46, wherein the relaxivity of the compound is about 0.06 to about 0.25.

48. The compound of any one of claim 4-30 or 43-47, wherein the relaxivity of the compound is about 0.072, about 0.14, or about 0.2.

49. The compound of any one of claims 1-30, wherein the relaxivity of the compound at pH 6.0 is about 1.2 to about 2.3 and the relaxivity at pH 7.4 is about 0.06 to about 0.25.

50. A composition comprising a compound of any one of claims 1-49, or a pharmaceutically acceptable salt or solvate thereof, and a pharmaceutically acceptable carrier.

51. The composition of claim 50, wherein the composition comprises a mixture of compounds of any one of claims 1-49, or a pharmaceutically acceptable salt or solvate thereof.

52. The composition of claim 50 or 51, wherein the composition is formulated for parenteral admiration.

53. The composition of any one of claims 50-52, wherein the composition is a solid formulated for dissolution in a pharmaceutically acceptable liquid medium prior to administration.

54. A method of magnetic resonance (MR) imaging a subject comprising:
(a) administering to a subject a compound of any one of claims 1-49 or a composition of any one of claims 50-53; and
(b) obtaining a magnetic resonance image of the subject after a period of time.

55. A method for imaging a blood clot in a subject comprising:
(a) administering to a subject a compound of any one of claims 1-49 or a composition of any one of claims 50-53; and
(b) obtaining a magnetic resonance image of the subject after a period of time.

56. A method for imaging a brain lesion in a subject comprising:
(a) administering to a subject a compound of any one of claims 1-49 or a composition of any one of claims 50-53; and
(b) obtaining a magnetic resonance image of the subject after a period of time.

57. A method for detecting the presence or absence of a solid tumor in a subject comprising:
(a) administering to a subject a compound of any one of claims 1-49 or a composition of any one of claims 50-53; and
(b) obtaining a magnetic resonance image of the subject after a period of time.

58. A method for determining the growth rate of a solid tumor in a subject having a solid tumor comprising:
(a) administering to the subject a compound of any one of claims 1-49 or a composition of any one of claims 50-53;
(b) obtaining a first magnetic resonance image of the subject after a period of time;
(c) administering to a subject a compound of any one of claims 1-49 or a composition of any one of claims 50-53 after a second period of time;
(d) obtaining a second magnetic resonance image of the subject after a period of time; and
(e) comparing the first magnetic resonance image of the subject and the second magnetic resonance image of the subject.

59. A method for detecting the presence or absence of a disrupted blood-brain-barrier in a subject comprising:
(a) administering to a subject a compound of any one of claims 1-49 or a composition of any one of claims 50-53;
(b) obtaining a first magnetic resonance image of the subject after a period of time;
(c) administering to a subject a compound of any one of claims 1-49 or a composition of any one of claims 50-53 after a second period of time;
(d) obtaining a second magnetic resonance image of the subject after a period of time; and
(e) comparing the first magnetic resonance image of the subject and the second magnetic resonance image of the subject.

60. A method for detecting the presence or absence of arterial stenosis in a subject comprising:
(a) administering to a subject a compound of any one of claims 1-49 or a composition of any one of claims 50-53;
(b) obtaining a first magnetic resonance image of the subject after a period of time;
(c) administering to a subject a compound of any one of claims 1-49 or a composition of any one of claims 50-53 after a second period of time;
(d) obtaining a second magnetic resonance image of the subject after a period of time; and
(e) comparing the first magnetic resonance image of the subject and the second magnetic resonance image of the subject.

61. A method for detecting the presence or absence of spinal stenosis in a subject comprising:
(a) administering to a subject a compound of any one of claims 1-49 or a composition of any one of claims 50-53;
(b) obtaining a first magnetic resonance image of the subject after a period of time;
(c) administering to a subject a compound of any one of claims 1-49 or a composition of any one of claims 50-53 after a second period of time;
(d) obtaining a second magnetic resonance image of the subject after a period of time; and
(e) comparing the first magnetic resonance image of the subject and the second magnetic resonance image of the subject.

62. The method of any one of claims 54-61, wherein the period of time is about 5 minutes to about 120 minutes.

63. The method of any one of claims 54-62, wherein the period of time is about 5 minutes to about 45 minutes.

64. The method of any one of claims 54-62, wherein the period of time is about 30 minutes to about 60 minutes.

65. The method of any one of claims 54-62, wherein the period of time is about 45 minutes to about 90 minutes.

66. The method of any one of claims 54-62, wherein the period of time is about 60 minutes to about 120 minutes.

67. The method of any one of claims 58-66, wherein the second period of time is about 2 weeks to about 24 months.

68. The method of any one of claims 58-66, wherein the second period of time is about 2 weeks to about 3 months.

69. The method of any one of claims 58-66, wherein the second period of time is about 2 months to about 6 months.

70. The method of any one of claims 58-66, wherein the second period of time is about 4 months to about 12 months.

71. The method of any one of claims 58-66, wherein the second period of time is about 8 months to about 18 months.

72. The method of any one of claims 58-66, wherein the second period of time is about 12 months to about 24 months.
